SEQUENCE LISTING (2 sequences)

SEQ ID NO: 1 - DNA, 40 bp, Artificial Sequence
  Capture probe
ccagtgaattcgagctcggtacccggggatcctctagagt

SEQ ID NO: 2 - DNA, 40 bp, Artificial Sequence
  Reporter Probe
cttagtctttagatgcaagcttggcgtaatcatggtcata

(19) United States
(12) Patent Application Publication
(10) Pub. No.: US 2005/0266476 A1
(43) Pub. Date: Dec. 1, 2005

(54) Dual bead assays including covalent linkages for improved specificity and related optical analysis discs

(76) Inventors: Brigitte Chau Phan, Fountain Valley, CA (US); Jorma Antero Virtanen, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/145,501

(22) Filed: Jun. 3, 2005

Related U.S. Application Data
(63) Continuation of application No. 10/038,297, filed on Jan. 4, 2002.
(60) Provisional application No. 60/271,922, filed on Feb. 27, 2001.
(60) Provisional application No. 60/259,806, filed on Jan. 4, 2001.

(51) Int. Cl. C12Q 1/68

(52) U.S. Cl. 435/6

(57) ABSTRACT

The invention provides compositions and methods for determining whether a target agent is present in a biological sample. The invention provides a sensitive and consumer friendly system for performing biological assays. More particularly, the invention is directed to the use of a biodisc in a dual bead assay designed to identify the presence of a target molecule. The invention further provides methods to optimize the selection of a suitable solid phase for use in a dual bead assay.

### CROSS REFERENCE TO RELATED APPLICATIONS

This application is a continuation of U.S. patent application Ser. No. 10/038,297, entitled DUAL BEAD ASSAYS INCLUDING COVALENT LINKAGES FOR IMPROVED SPECIFICITY AND RELATED OPTICAL ANALYSIS DISCS, filed Jan. 4, 2002, which is a nonprovisional application of and claims priority under 35 U.S.C. § 119(e) to the following two provisional patent applications: U.S. Provisional Patent Application No. 60/271,922, entitled METHODS FOR ATTACHING CAPTURE DNA AND REPORTER DNA TO SOLID PHASE INCLUDING SELECTION OF BEAD TYPE AS SOLID PHASE, filed Feb. 27, 2001 and U.S. Provisional Patent Application No. 60/259,806, entitled DEVICE AND METHODS FOR PERFORMING QUALITATIVE AND QUANTITATIVE ANALYSIS ON OPTICAL DISC PLATFORM, filed Jan. 4, 2001. The disclosure of each of the foregoing applications is incorporated herein by reference in its entirety.

### BACKGROUND OF THE INVENTION

The present invention relates to biological analysis and optical biodiscs.

There is a significant need to make diagnostic assays and forensic assays of all types faster and more local to the end-user. Ideally, clinicians, patients, investigators, the military, other health care personnel and consumers should be able to test themselves for the presence of certain factors or indicators in their systems, for the presence of certain biological material at a crime scene or on a battlefield. At present, there are a number of silicon based chips with nucleic acids and/or proteins attached thereto which are commercially available or under development. These chips are not for use by the end-user, or for use by persons or entities lacking very specialized expertise and expensive equipment.

U.S. Pat. No. 6,030,581, issued Feb. 29, 2000 (the '581 patent) is hereby incorporated by reference in its entirety. The '581 patent discloses an apparatus that includes an optical disc, adapted to be read by an optical reader, which has a sector having substantially self-contained assay system useful for localizing and detecting an analyte suspected of being in a sample. U.S. Pat. No. 5,993,665, issued Nov. 30, 1999 (the '665 patent) entitled “Quantitative Cell Analysis Methods Employing Magnetic Separation” discloses analysis of biological specimens in a fluid medium where the specimens are rendered magnetically responsive by immuno-specific binding with ferromagnetic colloid. The '665 patent is hereby incorporated by reference in its entirety.

### SUMMARY OF THE INVENTION

The present invention relates to performing assays, and particularly to using dual bead structures on a disc. The invention includes methods for preparing assays, methods for performing assays, discs for performing assays, and detection systems.

In one aspect, the present invention includes methods for determining whether a target agent is present in a biological sample. These methods can include mixing capture beads, each having at least one transport probe, reporter beads, each having at least one signal probe, and a biological sample. These components are mixed under binding conditions that permit formation of a dual bead complex if the target agent is present in the sample. The dual bead complex thus includes a reporter bead and a capture bead each bound to the target agent. The dual bead complex is isolated from the mixture to obtain an isolate. The isolate is then exposed to a capture field on an optical disc. The capture field has a capture agent that binds specifically to the signal probe or transport probe of the dual bead complex. The dual bead complex in the optical disc is then detected to indicate that the target agent is present in the sample, and if desired to indicate a concentration.

The capture beads can have a specified size and have a characteristic that makes them “isolatable.” The capture beads are preferably magnetic, in which case the isolating of dual bead complex (and some capture beads not part of a complex) in a mixture includes subjecting the mixture to a magnetic field with a permanent magnet or an electromagnet. Capture beads that are not magnetic may be isolated by centrifugal forces.

The reporter bead should have characteristics that make it identifiable and distinguishable with detection. The reporter beads can be made of one of a number of materials, such as latex, gold, plastic, steel, or titanium, and should have a known and specified size. The reporter beads can be fluorescent and can be yellow, green, red, or blue.

The dual bead complex can be formed on the disc itself, or outside the disc and added to the disc. To form the dual bead complex off the disc, methods referred to here as “one-step” or “two-step” can be employed. In the two-step method, the mixture initially includes capture beads and the sample. The capture beads are then isolated to wash away unbound sample and leaving bound and unbound capture beads in a first isolate. Reporter beads are then added to the first isolate to produce dual bead complex structures and the isolation process is repeated. The resulting isolate leaves dual bead complex with reporters, but also includes unbound capture beads without reporters. The reporters make the dual bead complex detectable.

In the “one-step” method, the capture beads, reporter beads and sample are mixed together from the start and then the isolation process isolates dual bead complex along with unbound capture beads.

These methods for producing and isolating dual bead complex structures can be performed on the disc. The sample and beads can be added to the disc together, or the beads can be pre-loaded on the disc so that only a sample needs to be added. The sample and beads can be added in a mixing chamber on the disc, and the disc can be rotated in one direction or in both to assist the mixing. An isolate can then be created, such as by applying an electromagnet and rotating to cause the material other than the capture beads to be moved to a waste chamber. The isolate is then directed through rotation to capture fields.

The dual bead complex structures can be detected on the capture field via various methods. In one embodiment, the detecting includes directing a beam of electromagnetic energy from a disc drive toward the capture field and analyzing electromagnetic energy returned from or transmitted past the reporter bead of the dual bead complex attached to the capture field. The disc drive assembly can include a detector and circuitry or software that senses the detector signal for a sufficient transition between light and dark (referred to as an “event”) to spot a reporter bead.

Beads can, alternatively, be detected based on their fluorescence. In this case, the energy source in the disc drive preferably has a wavelength controllable light source and a detector that is or can be made specific to a particular wavelength. Alternatively, a disc drive can be made with a specific light source and detector to produce a dedicated device, in which case the source may only need fine tuning.

The biological sample can include blood, serum, plasma, cerebrospinal fluid, breast aspirate, synovial fluid, pleural fluid, perintoneal fluid, pericardial fluid, urine, saliva, amniotic fluid, semen, mucus, a hair, feces, a biological particulate suspension, a single-stranded or double-stranded nucleic acid molecule, a cell, an organ, a tissue, or a tissue extract, or any other sample that includes a target that may be bound through chemical or biological processes.

In addition to these medical uses, the embodiments of the present invention can be used in other ways, such as for testing for impurities in a sample, such as food or water, or for otherwise detecting the presence of a material, such as a biological warfare agent.

The target agent can include, for example, a nucleic acid (such as DNA or RNA) or a protein (such as an antigen or an antibody). If a nucleic acid, both the transport probe and the signal probe can be a nucleic acid molecule complementary to the target nucleic acid. If a protein, both the transport probe and the signal probe can be an antibody that specifically binds the target protein.

The transport probe or signal probe can bind specifically to the capture agent on the optical disc due to a high affinity between the probe and the capture agent. This high affinity can, for example, be the result of a strong protein-protein affinity (i.e., antigen-antibody affinity), or the result of a complementarity between two nucleic acid molecules.

Preferably the binding is to the signal probe, and then the disc is rotated to move unbound structures, including capture beads not bound to reporter beads, away from the capture field. If the binding is to the transport probe, unbound capture beads will be included, although the reporter beads are still the beads that are detected. This may be acceptable if the detection is for producing a yes/no answer, or if a fine concentration detection is not otherwise required.

The transport probe and signal probe can each be one or more probes selected from the group consisting of single-stranded DNA, double-stranded DNA, single-stranded RNA, peptide nucleic acid, biotin, streptavidin, an antigen, an antibody, a receptor protein and a ligand. In a further embodiment, each transport probe comprises double-stranded DNA and single-stranded DNA, wherein the double-stranded DNA is proximate to the capture layer of the optical disc and the single-stranded DNA is distal relative to the capture layer of the optical disc.

The reporter bead and/or signal probe can be biotinylated and the capture agent can include streptavidin or neutravidin. Chemistry for affixing capture agents to the capture layer of the optical disc are generally known, especially in the case of affixing a protein or nucleic acid to solid surfaces. The capture agent can be affixed to the capture layer via an amino group or a thiol group.

The target agent can include a nucleic acid characteristic of a disease, or a nucleotide sequence specific for a person or having a nucleotide sequence specific for an organism, which may be a bacterium, a virus, a mycoplasm, a fungus, a plant, or an animal. The target agent can include a nucleic acid molecule associated with cancer in a human. The target nucleic acid molecule can include a nucleic acid which is at least a portion of a gene selected from the group consisting of: HER2neu, p52, p53, p21 and bcl-2. The target agent can be an antibody which is present only in a subject infected with HIV-1, a viral protein antigen, or a protein characteristic of a disease state in a subject. The methods and apparatus can be used for determining whether a subject is infected by a virus, whether nucleic acid obtained from a subject exhibits a single nucleotide mutation (SNM) relative to corresponding wild-type nucleic acid sequence, or whether a subject expresses a protein of interest, such as a bacterial protein, a fungal protein, a viral protein, an HIV protein, a hepatitis C protein, a hepatitis B protein, or a protein known to be specifically associated with a disease.

In another aspect, the invention includes multiplexing methods whereby more than one target agent (e.g., tens, hundreds, or even thousands of different target agents) can be identified on one optical disc. Multiple capture agents can be provided in a single chamber together in capture fields, or separately in separate capture fields. Different reporter beads can be used to be distinguishable from each other, such as beads that fluoresce at different wavelengths or different size reporter beads.

In another aspect, the invention includes an optical disc with a substrate, a capture layer over the substrate, and a capture agent bound to the capture layer, such that the capture agent binds to a dual bead complex. Multiple different capture agents can be used for different types of dual bead complexes. The disc can be designed to allow for some dual bead processing on the disc with appropriate chambers and fluidic structures, and can be pre-loaded with reporter and capture beads so that only a sample needs to be added to form the dual bead complex structures.

In yet another aspect, the invention includes a disc and disc drive system for performing dual bead assays. The disc drive can include an electromagnet for performing the isolation process, and may include appropriate light source control and detection for the type of reporter beads used. The disc drive can be optical or magneto-optic.

For processing performed on the disc, the drive can include an electromagnet, the disc preferably has a mixing chamber, a waste chamber, and capture area. The sample is mixed with beads in the mixing chamber, a magnetic field is applied over the mixing chamber, and the sample not held by the magnet is directed to the waste chamber so that all magnetic beads, whether bound into a dual bead complex or unbound, remain in the mixing chamber. The magnetic beads are then directed to the capture area. One of a number of different valving arrangements can be used to control the flow. In still another aspect of the present invention, a biodisc is produced for use with biological samples and is used in conjunction with a disc drive, such as a magneto-optical disc drive, that can form magnetic regions on a disc. In a magneto-optical disc and drive, magnetic regions can be formed in a highly controllable and precise manner. These regions can be used to magnetically bind magnetic beads, including unbound magnetic capture beads or including dual bead complexes with magnetic capture beads. The magneto-optical disc drive can write to selected locations on the disc, and then use an optical reader to detect features located at those regions. The regions can be erased, thereby allowing the beads to be released.

In still another aspect, the invention includes a method for use with a biodisc and drive including forming magnetic regions on the biodisc, and providing magnetic beads to the discs so that the beads bind at the magnetic locations. The method preferably further includes detecting at the locations where the magnetic beads bind biological samples, preferably using reporter beads that are detectable, such as by fluorescence or optical event detection. The method can be formed in multiple stages in terms of time or in terms of location through the use of multiple chambers. The regions are written to and a sample is moved over the magnetic regions in order to capture magnetic beads. The regions can then be erased and released if desired. This method allows many different tests to be performed at one time, and can allow a level of interactivity between the user and the disc drives such that additional tests can be created during the testing process.

In another aspect, the invention provides a method of evaluating a solid phase for use in a dual bead assay, the method comprising selecting a test solid phase, binding a probe to the test solid phase in the presence or absence of a cross-linking agent, determining the total amount of probe bound to the test solid phase in the presence or absence of a cross-linking agent, determining the percentage of probe bound covalently to the solid phase, determining the amount of probe bound to the solid phase covalently, and calculating the percentage of probe bound covalently to the solid phase, wherein if no less than approximately 80% of the probe is bound covalently, the solid phase is suitable for use in a dual bead assay.

In certain embodiments thereof, the solid phase is a bead, particularly a magnetic bead. In other embodiments thereof, the solid phase is a surface on a biodisc. Probes that may be tested for binding to a particular solid phase include, but are not limited to, nucleic acids and proteins. In addition, various embodiments of this aspect of the invention utilize probe comprising a linker molecule.

The apparatus and methods in embodiments of the present invention can be designed for use by an end-user, inexpensively, without specialized expertise and expensive equipment. The system can be made portable, and thus usable in remote locations where traditional diagnostic equipment may not generally be available. Other features and advantages will become apparent from the following detailed description, drawings, and claims.

### BRIEF DESCRIPTION OF THE DRAWINGS

FIG. 1 is a perspective view of an optical disc system according to the present invention.

FIGS. 2A, 2B, and 2C are respective exploded, top, and perspective views of a reflective disc according to embodiments of the present invention.

FIGS. 3A, 3B, and 3C are respective exploded, top, and perspective views of a transmissive disc according to embodiments of the present invention.

FIG. 4 is a block diagram and pictorial diagram of an optical reading system according to embodiments of the present invention.

FIGS. 5 and 6 are cross sectional views of discs according to embodiments of the present invention.

FIGS. 7-10 are schematic representations of a capture bead, a reporter bead, and a dual bead complex.

FIGS. 11 and 12 are pictorial representations of methods for producing dual bead complex solutions.

FIGS. 13A-13D and 14A-14B are partial cross sectional views of a dual bead complex binding to a capture layer over a substrate of an optical disc according to the present invention.

FIGS. 15A-15D illustrate methods according to the present invention for detecting the presence of target DNA in a sample of DNA.

FIGS. 16A-16B and 17A-17B are diagrams and graphs showing a signal detected from a reporter bead and a capture bead separately and as bound, showing that a 2.1 micron reporter bead is distinguishable from a 3 micron capture bead and that the complex is detectable.

FIG. 18 is a bar graph showing multiplexed results according to the present invention.

FIG. 19 is a schematic representation of combining beads for dual bead assay multiplexing according to embodiments of the present invention.

FIG. 20A is a graph showing a standard curve demonstrating the detection limit for fluorescent beads detected with a fluorimeter.

FIG. 20B is a graphic display demonstrating the lower limit of target detection for a fluorescent reporter bead as measured with a fluorimeter.

FIG. 20C is a pictorial demonstrating that the sensitivity of a dual bead assay quantified with a CD reader is as little as one reporter molecule.

FIG. 21 is a schematic representation of a fluidic circuit according to the present invention.

FIGS. 22A-22C and 23A-23C are schematics of fluidic circuits that implement the structure of FIG. 21 according to the present invention.

FIGS. 24 and 25A-25C are a perspective view and top views of a disc wherein the fluid control includes a configuration of chambers and passages on a disc according to the present invention.

FIG. 26 is a perspective view of a magneto-optical biodisc with magnetic regions and capture beads and dual bead complexes bound thereto, according to the present invention.

FIG. 27 is a schematic presenting a method for evaluating a solid phase for covalent conjugation of a probe.

FIG. 28 is a schematic detailing various steps in the quantification of covalently-bound and non-covalently bound probe to a solid substrate.

FIG. 29A is a graphic presentation of experimental results of various testings of magnetic bead carriers for covalent linkage of a probe.

FIG. 29B is a graphic presentation of experimental results of various testings of fluorescent bead carriers for covalent linkage of a probe.

FIG. 30A is a pictorial demonstrating the structural differences between single-stranded and double-stranded DNA that are relevant to their use as probes.

FIG. 30B is a graphic presentation of results of an experiment designed to evaluate the binding properties of single-stranded and double-stranded DNA to a solid phase.

FIG. 31A is a graphic presentation of enzyme assay results of a screen of two different magnetic beads for use in a dual bead assay. These results indicate that both of the tested beads bind a similar amount of target regardless of whether the probe is bound covalently or non-covalently.

FIG. 31B is a graphic presentation of results of a screen designed to examine the number of reporter beads captured by two different capture beads. These results indicate that covalent bonding of the probe to the capture bead greatly improves assay sensitivity.

FIG. 32 is a graphic presentation demonstrating that the introduction of PEG linkers into probes significantly improves target mediated binding.

### DETAILED DESCRIPTION

Optical Disc and Drive System

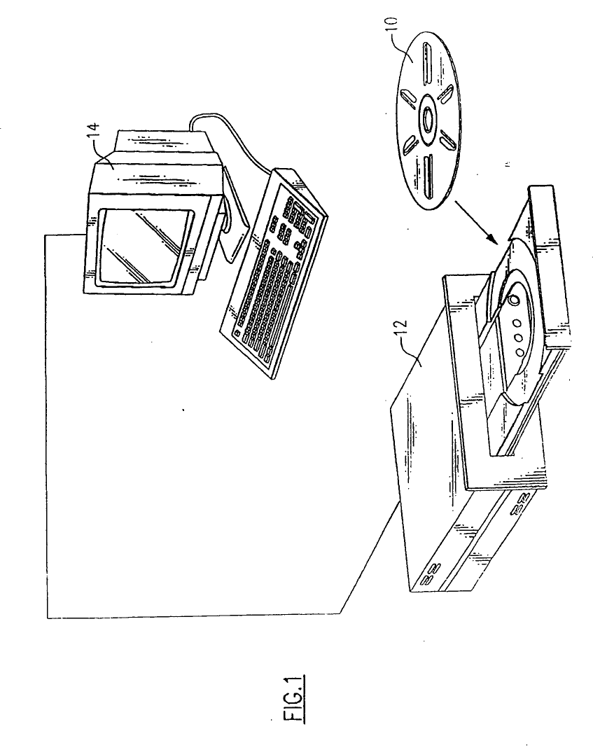

FIG. 1 is a perspective view of an optical disc 10 shown for insertion in an optical disc drive 12. Drive 12, in conjunction with software in the drive or associated with a separate computer, can cause images, graphs, or output data to be displayed on display monitor 14. As indicated below, there are different types of discs and drives that can be used. The disc drive can be in a unit separate from a controlling computer, or provided in a bay within a computer. The device can be made as portable as a laptop computer, and thus usable with battery power and in remote locations not generally served by advanced diagnostic equipment. The drive is preferably a conventional drive with minimal or no hardware modification, but can be a dedicated biodisc drive.

Optical disc 10 for use with embodiments of the present invention may have any suitable shape, diameter, or thickness, but preferably is implemented on a round disc with a diameter and a thickness similar to those of a compact disc (CD), a recordable CD (CD-R), CD-RW, a digital versatile disc (DVD), DVD-R, DVD-RW, or other standard optical disc format. The disc may include encoded information, preferably in a known format, for performing, controlling, and post-processing a test or assay, such as information for controlling the rotation rate and direction of the disc, timing for rotation, stopping and starting, delay periods, locations of samples, position of the light source, and power of the light source. Such encoded information is referred to generally here as operational information.

The disc may be referred to as reflective, transmissive, or some combination of reflective and transmissive. In a reflective disc, an incident light beam is focused onto the disc (typically at a reflective surface where information is encoded), reflected, and returned through optical elements to a detector on the same side of the disc as the light source. In a transmissive disc, light passes through the disc (or portions thereof) to a detector on the other side of the disc from the light source. In a transmissive portion of a disc, some light may also be reflected and detected as reflected light.

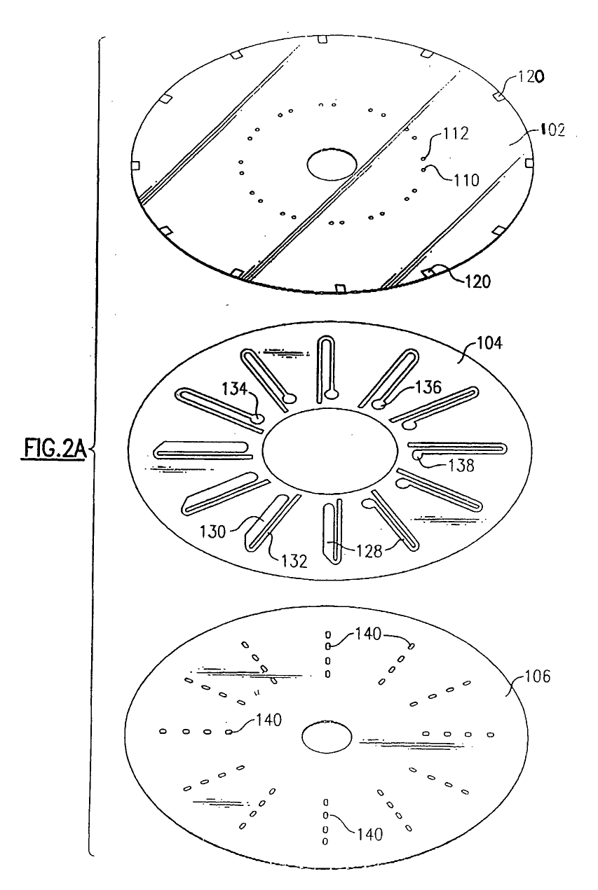

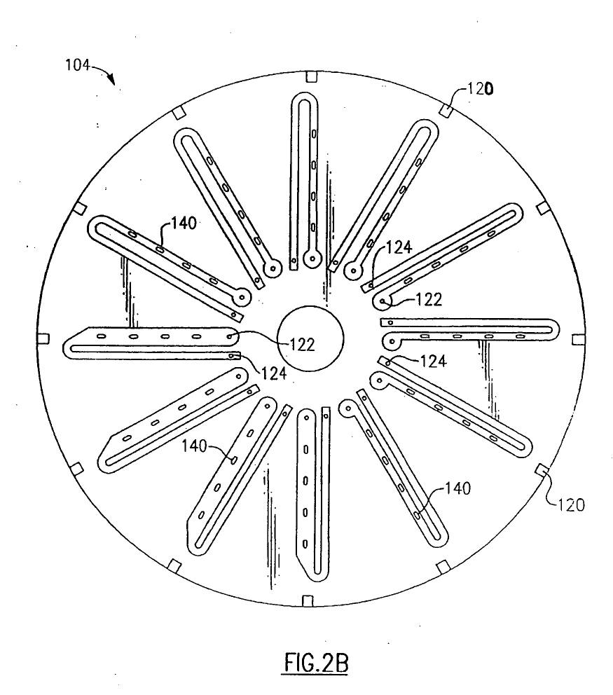

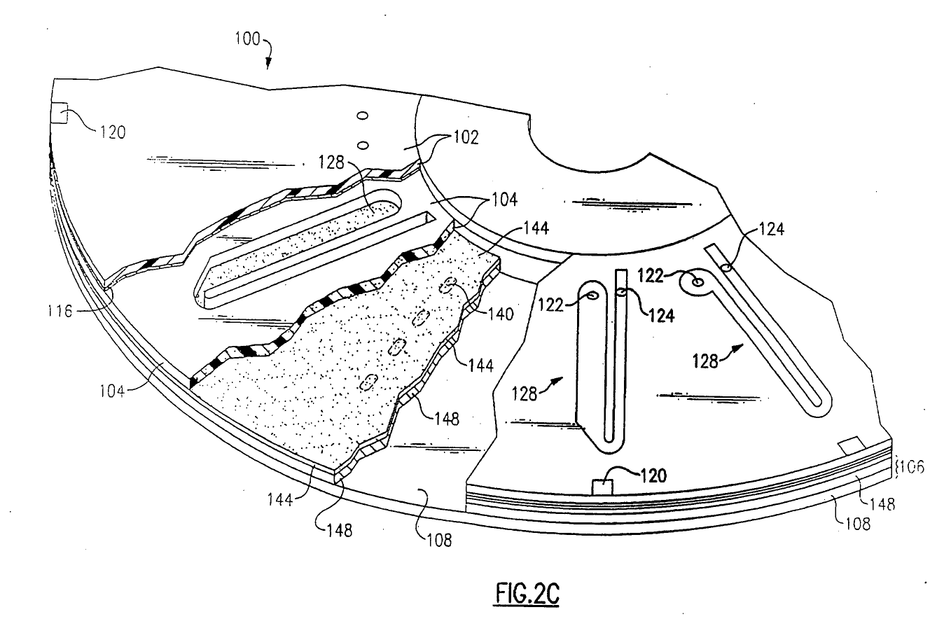

Referring to FIGS. 2A, 2B, and 2C, a reflective disc 100 is shown with a cap 102, a channel layer 104, and a substrate 106. Cap 102 has inlet ports 110 for receiving samples and vent ports 112. Cap 102 may be formed primarily from polycarbonate, and may be coated with a reflective layer 116 on the bottom thereof. Reflective layer 116 is preferably made from a metal, such as aluminum or gold, and is used to encode the operational information.

Channel layer 104 defines fluidic circuits 128 by having desired shapes cut out from channel layer 104. Each fluidic circuit 128 preferably has a flow channel 130 and a return vent channel 132, and some have a mixing chamber 134. A mixing chamber 136 can be symmetrically formed relative to the flow channel 130, while an off-set mixing chamber 138 is formed to one side of the flow channel 130. Fluidic circuits 128 are rather simple in construction, but a fluidic circuit can include other channels and chambers, such as preparatory regions or a waste region, as shown, for example, in U.S. Pat. No. 6,030,581, which is incorporated herein by reference, and can include valves and other fluid control structures. Channel layer 104 can include adhesives for bonding to the substrate and to the cap.

Substrate 106 has polycarbonate layer 108, and has target zones 140 formed as openings in a reflective layer 148 deposited on the top of layer 108. Target zones 140 may be formed by removing portions of reflective layer 148 in any desired shape, or by masking target zone areas before applying reflective layer 148. Reflective layer 148 is preferably formed from a metal, such as aluminum or gold, and can be configured with the rest of the substrate to encode operational information that is read with incident light, such as through a wobble groove or through an arrangement of pits. Light incident from under substrate 106 thus is reflected by layer 148, except at target zones 140, where it is reflected by layer 116. Target zones are where investigational features are detected. If the target zone is a location where an antibody, strand of DNA, or other material that can bind to a target is located, the target zone can be referred to as a capture zone.

Referring particularly to FIG. 2C, optical disc 100 is cut away to illustrate a partial cross-sectional view. An active capture layer 144 is formed over reflective layer 148. Capture layer 144 may generally be formed from nitrocellulose, polystyrene, polycarbonate, gold, activated glass, modified glass, or a modified polystyrene, for example, polystyrene-co-maleic anhydride. Channel layer 104 is over capture layer 144.

Trigger marks 120 are preferably included on the surface of a reflective layer 148, and may include a clear window in all three layers of the disc, an opaque area, or a reflective or semi-reflective area encoded with information. In operation, samples can be introduced through inlet ports 110 of cap 102. When rotated, the sample moves outwardly from inlet port 110 along capture layer 144. Through one of a number of biological or chemical reactions or processes, detectable features, referred to as investigational features, may be present in the target zones. Examples of such processes are shown in the incorporated U.S. Pat. No. 6,030,581.

The investigational features captured by the capture layer with a capture agent may be designed to be located in the focal plane coplanar with reflective layer 148, where an incident beam is typically focused in conventional readers; alternatively, the investigational features may be captured in a plane spaced from the focal plane. The former configuration is referred to as a “proximal” type disc, and the latter a “distal” type disc.

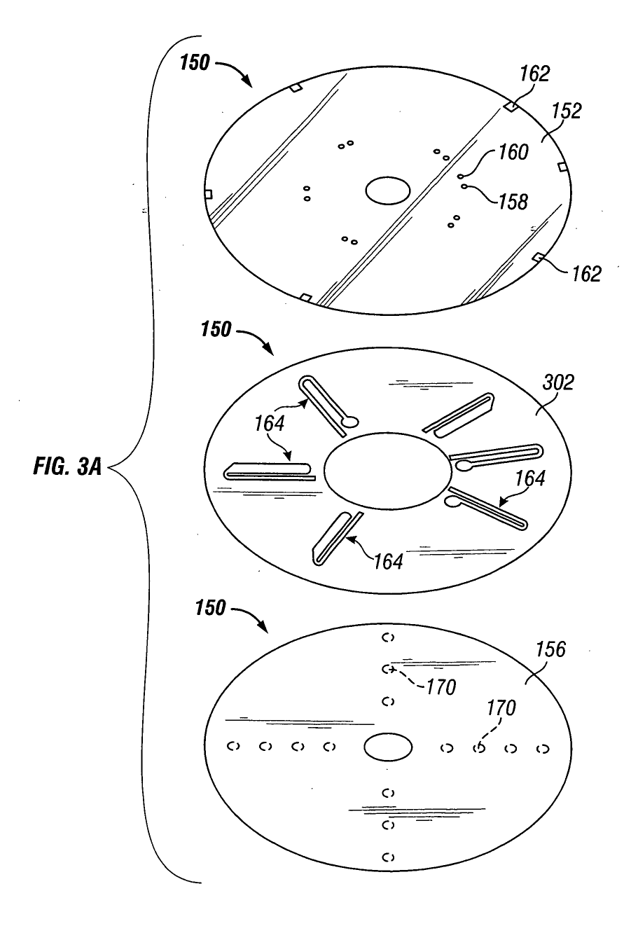

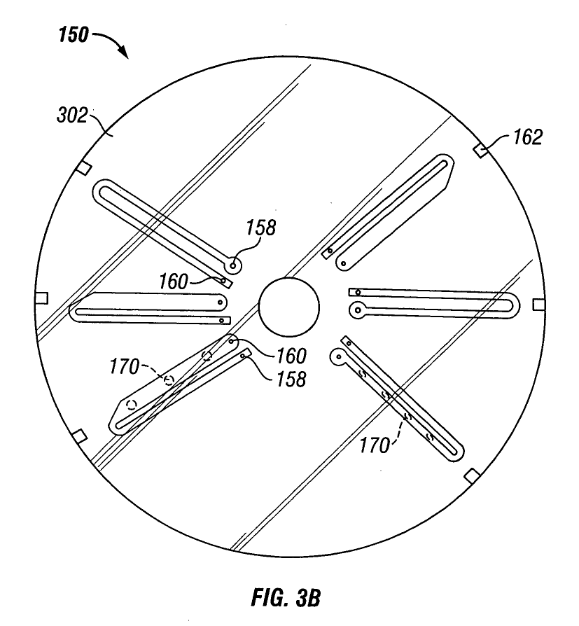

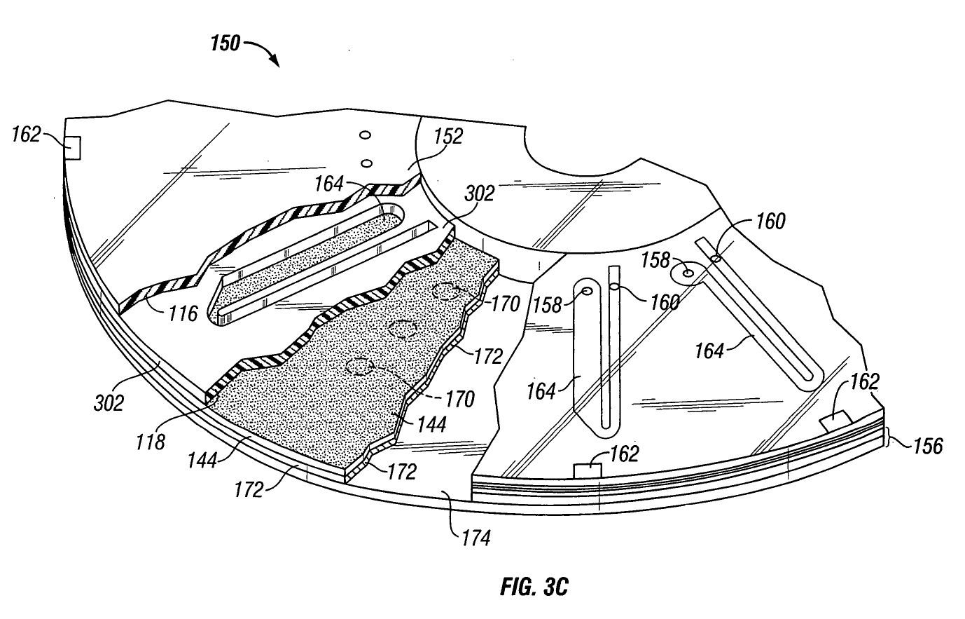

Referring to FIGS. 3A, 3B, and 3C, a transmissive optical disc 150 has a cap 152, a channel layer 302, and a substrate 156. Cap 152 includes inlet ports 158 and vent ports 160 and is preferably formed mainly from polycarbonate. Trigger marks 162, similar to those for reflective disc 100, may be included. Channel layer 302 has fluidic circuits 164, which can have structure and use similar to those described in conjunction with FIGS. 2A, 2B, and 2C. Substrate 156 may include target zones 170, and preferably includes a polycarbonate layer 174. Substrate 156 may, but need not, have a thin semi-reflective layer 172 deposited on top of layer 174. Semi-reflective layer 172 is preferably significantly thinner than reflective layer 148 on substrate 106 of reflective disc 100 (FIGS. 2A-2C). Semi-reflective layer 172 is preferably formed from a metal, such as aluminum or gold, but is sufficiently thin to allow a portion of an incident light beam to penetrate and pass through layer 172, while some of the incident light is reflected back. A gold film layer, for example, is 95% reflective at a thickness greater than about 700 Å, while the transmission of light through the gold film is about 50% transmissive at approximately 100 Å.

FIG. 3C is a cut-away perspective view of transmissive disc 150. The semi-reflective nature of layer 172 makes its entire surface potentially available for target zones, including virtual zones defined by trigger marks or encoded data patterns on the disc. Target zones 170 may also be formed by marking the designated area in the indicated shape or alternatively in any desired shape. Markings to indicate target zone 170 may be made on semi-reflective layer 172 or on a bottom portion of substrate 156 (under the disc). Target zones 170 may be created by silk screening ink onto semi-reflective layer 172.

An active capture layer 180 is applied over semi-reflective layer 172. Capture layer 180 may be formed from the same materials as described above in conjunction with layer 144 (FIG. 2C) and serves substantially the same purpose when a sample is provided through an opening in disc 150 and the disc is rotated. In transmissive disc 150, there is no reflective layer comparable to reflective layer 116 in reflective disc 100 (FIG. 2C).

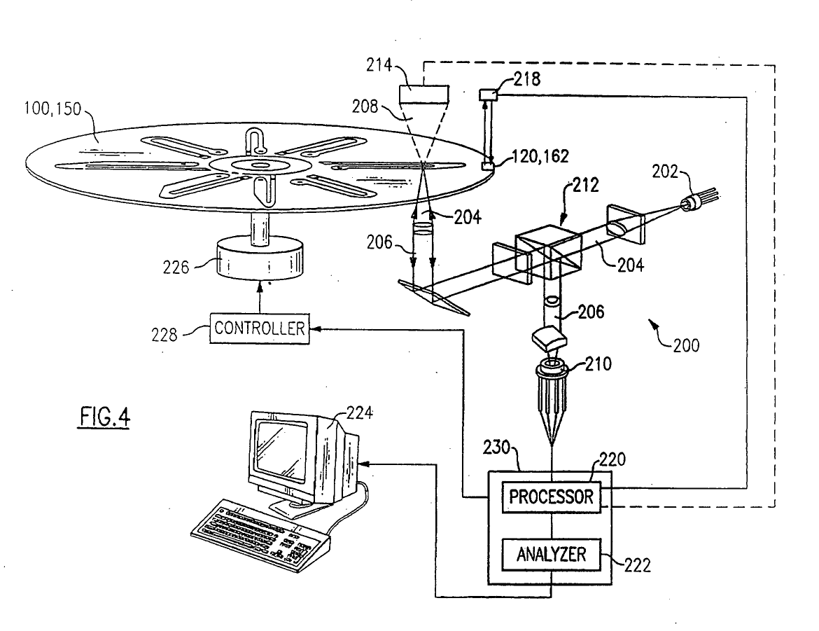

FIG. 4 shows an optical disc reader system 200. This system may be a conventional reader for CD, CD-R, DVD, or other known comparable format, a modified version of such a drive, or a completely distinct dedicated device. The basic components are a motor for rotating the disc, a light system for providing light, and a detection system for detecting light.

A light source 202 provides light to optical components 212 to produce an incident light beam 204. In the case of reflective disc 100, a return beam 206 is reflected from either reflective surface 148 or 116. Return beam 206 is provided back to optical components 212, and then to a bottom detector 210. For transmissive disc 150, a transmitted beam 208 is detected by a top detector 214. Optical components 212 can include a lens, a beam splitter, and a quarter wave plate that changes the polarization of the light beam so that the beam splitter directs a reflected beam through the lens to focus the reflected beam onto the detector. An astigmatic element, such as a cylindrical lens, may be provided between the beam splitter and detector to introduce astigmatism in the reflected light beam. The light source can be controllable for wavelength and power in response to data introduced by the user or read from the disc. This control is especially useful if it is desired to detect multiple different structures that fluoresce at different wavelengths.

Data from detector 210 and/or detector 214 is provided to a computer 230 including a processor 220 and an analyzer 222. An image or output results can then be provided to a monitor 224. Computer 230 can represent a desktop computer, programmable logic, or some other processing device, and also can include a connection (such as over the Internet) to other processing and/or storage devices. A drive motor 226 and a controller 228 are provided for controlling the rotation and direction of disc 100 or 150. Controller 228 and the computer 230 with processor 220 can be in communication or can be the same computer. Methods and systems for reading such a disc are also shown in Gordon, U.S. Pat. No. 5,892,577, which is incorporated herein by reference.

The detector can be designed to detect all light that reaches the detector, or though its design or an external filter, light only at specific wavelengths. By making the detector controllable in terms of the detectable wavelength, beads or other structures that fluoresce at different wavelengths can be separately detected.

A hardware trigger sensor 218 may be used with either a reflective or transmissive disc. Triggering sensor 218 provides a signal to computer 230 (or to some other electronics) to allow for the collection of data by processor 220 only when incident beam 204 is on a target zone. Alternatively, software read from a disc can be used to control data collection by processor 220 independent of any physical marks on the disc.

The substrate layer may be impressed with a spiral track that starts at an innermost readable portion of the disc and then spirals out to an outermost readable portion of the disc. In a non-recordable CD, this track is made up of a series of embossed pits with varying length, each typically having a depth of approximately one-quarter the wavelength of the light that is used to read the disc. The varying lengths and spacing between the pits encode the operational data. The spiral groove of a recordable CD-like disc has a detectable dye rather than pits. This is where the operation information, such as the rotation rate, is recorded. Depending on the test, assay, or investigational protocol, the rotation rate may be variable with intervening or consecutive periods of acceleration, constant speed, and deceleration. These periods may be closely controlled both as to speed and time of rotation to provide, for example, mixing, agitation, or separation of fluids and suspensions with agents, reagents, antibodies, or other materials.

Numerous designs and configurations of an optical pickup and associated electronics may be used in the context of the embodiments of the present invention. Further details and alternative designs for compact discs and readers are described in Compact Disc Technology, by Nakajima and Ogawa, IOS Press, Inc. (1992); The Compact Disc Handbook, Digital Audio and Compact Disc Technology, by Baert et al. (eds.), Books Britain (1995); and CD-Rom Professional's CD-Recordable Handbook: The Complete Guide to Practical Desktop CD, Starrett et al. (eds.), ISBN: 0910965188 (1996); all of which are incorporated herein in their entirety by reference.

The disc drive assembly is thus employed to rotate the disc, read and process any encoded operational information stored on the disc, analyze the liquid, chemical, biological, or biochemical investigational features in an assay region of the disc, to write information to the disc either before or after the material in the assay zone is analyzed by the read beam of the drive or deliver the information via various possible interfaces, such as Ethernet to a user, database, or anywhere the information could be utilized.

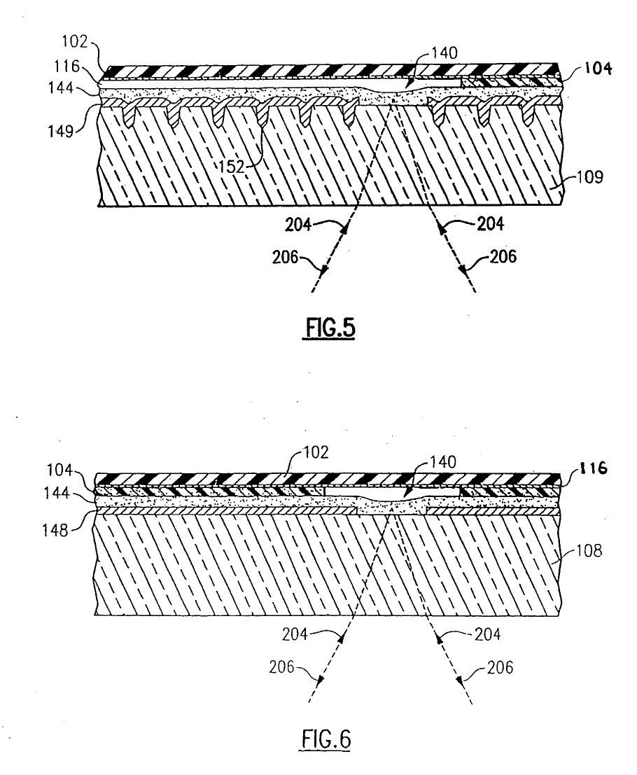

FIGS. 5 and 6 are partial cross sectional views of embodiments of reflective discs that can be used according to the present invention. In each case, there is a substrate 108, 109 and a reflective layer 148, 149. An capture layer 144 is over the reflective layer. A capture field 140 is formed by removing an area or portion of the reflective layer at a desired location or, alternatively, by masking the desired area prior to applying the reflective layer. A plastic adhesive member 104 with cut-out shapes to define channels is applied over the capture layer. A cap portion 102 with a second reflective layer 116 is applied over adhesive member 136 to form a flow channel 320. In each case, the incident light is at 204 and the reflected light is at 206.

Substrate 146 in FIG. 5 includes a series of wobble grooves 152, which are typical part of a CD-R disc. Grooves 152 are in the form of a spiral extending from near the center of the disc toward the outer edge, and are implemented so that an incident beam can track long the spiral. The spiral groove in a CD-R disc contains a dye, rather than pits and lands which are typically employed in a prerecorded CD. The reflective layer applied over the grooves in this embodiment is, as illustrated, conformal in nature. The embodiment of FIG. 6, by contrast, does not include a wobble groove, and thus is more similar to a CD.

Dual Bead Complex

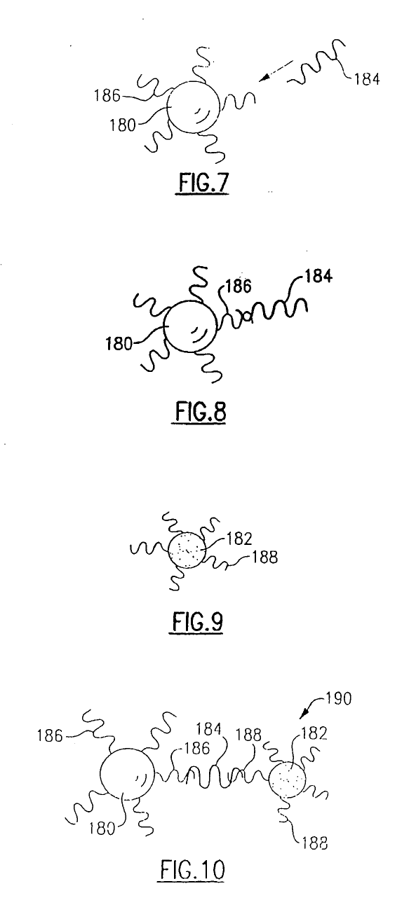

FIGS. 7-10 show a capture bead 180, a reporter bead 182, and the formation of a dual bead complex 190.

Capture bead 180 can be used in conjunction with a variety of different assays including biological assays such as immunoassays, molecular assays, and more specifically genetic assays. In the case of immunoassays, transport probes 186 are conjugated onto the beads. Transport probes 186 would include proteins, such as antigens or antibodies, implemented to capture protein targets. In the case of molecular assays, the transport probe would include nucleic acids such as DNA or RNA implemented to capture genetic targets.

A target agent 184, shown here as target DNA or RNA from a test sample, is added to a capture bead 180 coated with transport probes 186. In this implementation, transport probes 186 are formed from desired nucleic acids.

Capture bead 180 has a characteristic that allows it to be isolated from a material suspension or solution. The capture bead may be selected based upon a desired size, and a preferred way to make it isolatable is for it to be magnetic.

FIG. 8 illustrates the binding of target agent 184 to complementary transport probes 186 on capture bead 180 in the genetic assay implementation of the present invention. In an immunoassay version hereof, transport probes 186 can alternatively include antibodies or antigens for binding to a target protein.

FIG. 9 shows a reporter bead 182 coated with signal probes 188 complementary to target agent 184 (see FIG. 8). Reporter bead 182 is selected based upon a desired size and the material properties for detection and reporting purposes, such as a 2.1 micron polystyrene bead. Signal probes 188 can be antigens or antibodies implemented to capture protein targets, or strands of DNA or RNA to capture target DNA.

FIG. 10 is a physical representation of a dual bead complex 190 that can be formed from capture bead 180 with probe 186, target agent 184, and reporter bead 182 with probe 188. Probes 186, 188 conjugated on capture bead 180 and reporter bead 182, respectively, have sequences complementary to the target agent 184, but not to each other.

Off-Disc Formation of Dual Bead Complex

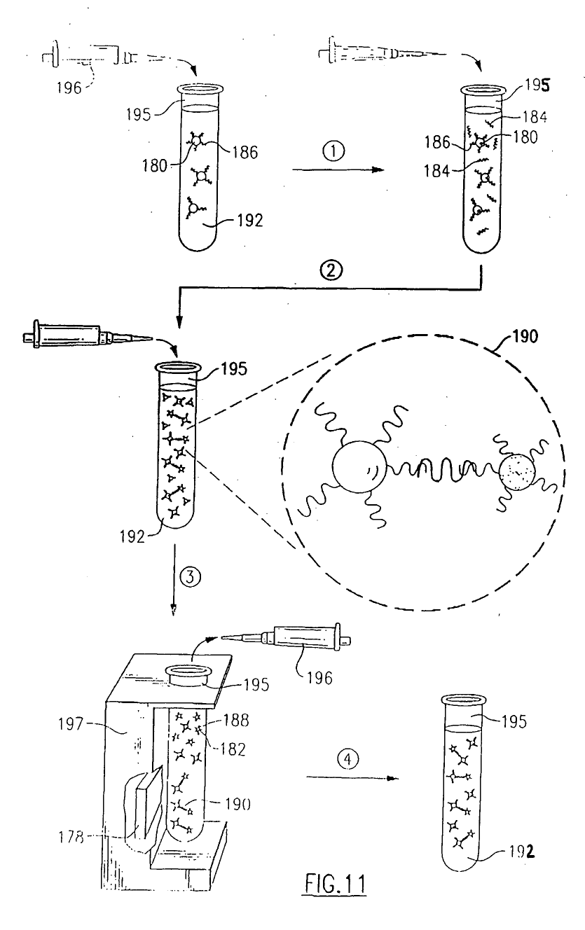

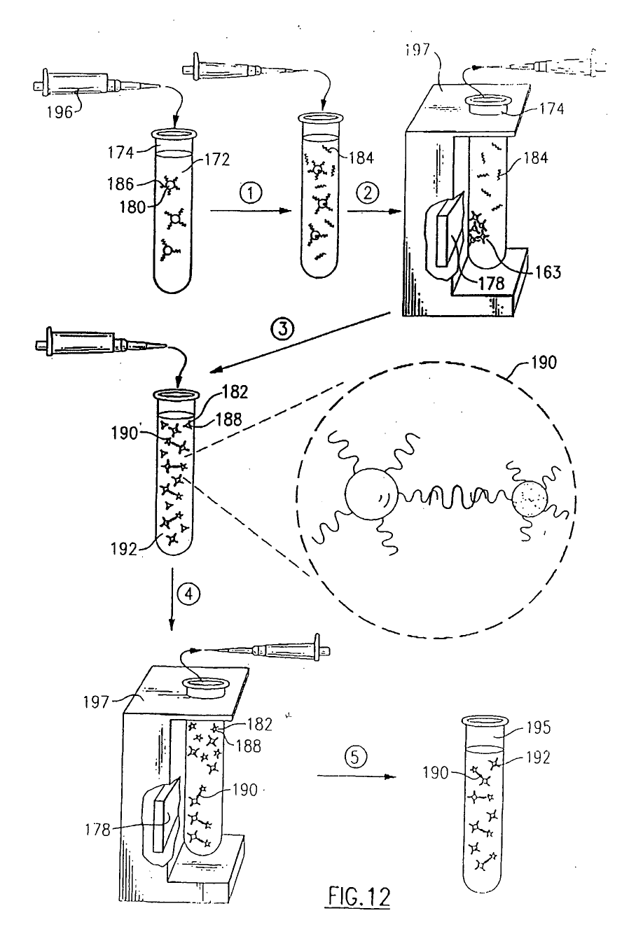

FIGS. 11 and 12 are detailed pictorial representations of methods for formation of dual bead complex outside of a disc, although the principles apply to on-disc formation as well.

FIG. 11 illustrates a “one-step” method to create dual bead complex structures in a solution. Capture beads 180, e.g., on the order of 10E+07 in number and each on the order of 1 micron or above in diameter, are coated with transport probes 186 complementary to a target agent (shown here as DNA, but others can be used) in a buffer solution 192. In one embodiment, capture agents which are complementary to a portion of the target agent are conjugated to 3 micron magnetic capture beads 180 via EDC conjugation. Capture beads 180 are suspended in hybridization solution and are loaded into a test tube 195 via injection with pipette 196. The preferred hybridization solution is composed of 0.2M NaCl, 10 mM MgCl₂, 1 mM EDTA, 50 mM Tris-HCl, pH 7.5, and 5× Denhart's mix. A desirable hybridization temperature is 37 degrees Celsius.

Target DNA is added to the solution. The target DNA binds to the complementary sequences of transport probe 186 attached to the capture bead 180 (see FIG. 8).

Reporter beads 182 are coated with signal probe 188 which are complementary to the target DNA and are added to the solution. In one embodiment, signal probes 188, which are complementary to a portion of the target DNA, are conjugated to 2.1 micron fluorescent reporter beads 182. Signal probes 188 and transport probes 186 each have sequences that are complementary to the target DNA, but not complementary to each other. After adding reporter beads 182, the dual bead complex 190 is formed such that the target DNA links capture bead 180 and reporter beads 182. With specific and thorough washing, there should be minimal non-specific binding between reporter bead 182 and capture bead 180. The target agent and signal probe 188 are allowed to hybridize for three to four hours at 37 degrees Celsius.

In this embodiment and others, it was found that intermittent mixing (i.e., periodically mixing and then stopping) produced greater yield of dual bead complex than continuous mixing during hybridization.

After hybridization, dual bead complex 190 is separated from unbound reporter beads in the solution. The solution can be exposed to a magnetic field to capture the dual bead complex structures 190 using the magnetic properties of capture bead 180. The magnetic field can be encapsulated in a magnetic test tube rack 197 with a built-in magnet 178, which can be permanent or electromagnetic to draw out the magnetic beads and remove any unbound reporter beads in the suspension. Note that capture beads not bound to reporter beads will also be isolated.

The purification process includes the removal of supernatant containing free-floating particles. Wash buffer is added into the test tube and the bead solution is mixed well. The preferred wash buffer for the one step assay consists of 145 mM NaCl, 50 mM Tris, pH 7.5, 0.1% SDS, 0.05% Tween, 0.25% NFDM, and 10 mM EDTA. Most of the unbound reporter beads 182, free-floating DNA, and non-specifically bound particles are agitated and removed from the supernatant. The dual bead complex can form a matrix of capture beads, target sequences, and reporter beads, wherein the wash process can further assist in the extraction of free floating particles trapped in the lattice structure of overlapping dual bead particles. Once the dual bead complex has been washed approximately 3-5 times with wash buffer solution, the assay mixture is loaded into the disc and ready to be analyzed.

This “one-step” dual bead assay and related method described in conjunction with FIG. 11 can be readily adapted to other assays including the immunoassays and general molecular assays discussed above which employ, alternatively, proteins such as antigens or antibodies implemented as the transport probes, target agents, and signal probes accordingly.

FIG. 12 shows an alternative method referred to here as a “two-step” isolation to create the dual bead complex. Capture beads 180 are coated with transport probes 186 complementary to target agent 184 (shown here as DNA) placed into a buffer solution 192. In this embodiment, transport probes 186 which are complementary to a portion of target agent 184 are conjugated to 3 micron magnetic capture beads 180 via EDC conjugation. Capture beads 180, suspended in hybridization solution, are loaded into a test tube 195 via injection from pipette 196. The preferred hybridization solution is composed of 0.2M NaCl, 10 mM MgCl₂, 1 mM EDTA, 50 mM Tris-HCl, pH 7.5, and 5× Denhart's mix. A desirable hybridization temperature is 37 degrees Celsius.

Target agent 184 is added to the solution and binds to the complementary sequences of transport probe 186 attached to capture bead 180. Target agent 184 and the transport probe 186 are allowed to hybridize for 2 to 3 hours at 37 degrees Celsius.

Target agents 184 bound to the capture beads are separated from unbound species in solution by exposing the solution to a magnetic field to isolate bound target sequences by using the magnetic properties of the capture bead 180. The magnetic field can be enclosed in a magnetic test tube rack 176 with a built-in magnet permanent 178 or electromagnet to draw out the magnetic beads and remove any unbound target DNA free-floating in the suspension via pipette extraction of the solution. A wash buffer is added and the separation process can be repeated. The preferred wash buffer after the transport probes 186 and target DNA hybridize, consists of 145 mM NaCl, 50 mM Tris, pH 7.5, and 0.05% Tween.

Reporter beads 182 are added to the solution as discussed in conjunction with the method shown in FIG. 11. Reporter beads 182 are coated with signal probes 188 which are complementary to target agent 184. Signal probes 188, which are complementary to a portion of target agent 184, are conjugated to 2.1 micron fluorescent reporter beads 182. Signal probes 188 and transport probes 186 each have sequences that are complementary to target agent 184, but not complementary to each other. After the addition of reporter beads 182, the dual bead complex structures 190 are formed. In this formation, target agent 184 links magnetic capture bead 180 and reporter bead 182. Using the preferred buffer solution, with specific and thorough washing, there is minimal non-specific binding between the reporter beads and the capture beads. Target agent 184 and signal probe 188 are allowed to hybridize for 2-3 hours at 37 degrees Celsius.

After hybridization, dual bead complex 190 is separated from unbound species in solution. The solution is again exposed to a magnetic field to isolate the dual bead complex 190 using the magnetic properties of the capture bead 180. Note again that the isolate will include capture beads not bound to reporter beads

A purification process to remove supernatant containing free-floating particles includes adding wash buffer into the test tube and mixing the bead solution well. The preferred wash buffer for the two step assay consists of 145 mM NaCl, 50 mM Tris, pH 7.5, 0.1% SDS, 0.05% Tween, 0.25% NFDM, and 10 mM EDTA. Most unbound reporter beads, free-floating DNA, and non-specifically bound particles are agitated and removed from the supernatant. The dual bead complex can form a matrix of capture beads, target agents, and reporter beads, wherein the wash process can further assist in the extraction of free floating particles trapped in the lattice structure of overlapping dual bead particles. Once the dual bead complex 190 has been washed approximately 3-5 times with wash buffer solution, the assay mixture is loaded into the disc and ready to be analyzed.

The two-step dual bead assay and related method described in conjunction with FIG. 12 can be readily adapted to other assays including the immunoassays and general molecular assays discussed above which employ, alternatively, proteins such as antigens or antibodies implemented as the transport probes, target agents, and signal probes accordingly.

Dual Bead Complex Binding on an Optical Disc

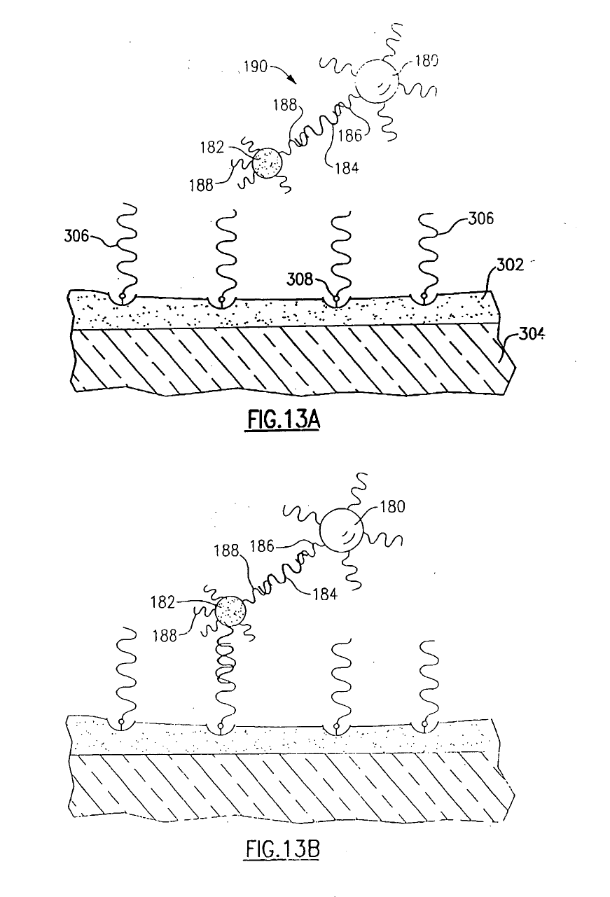

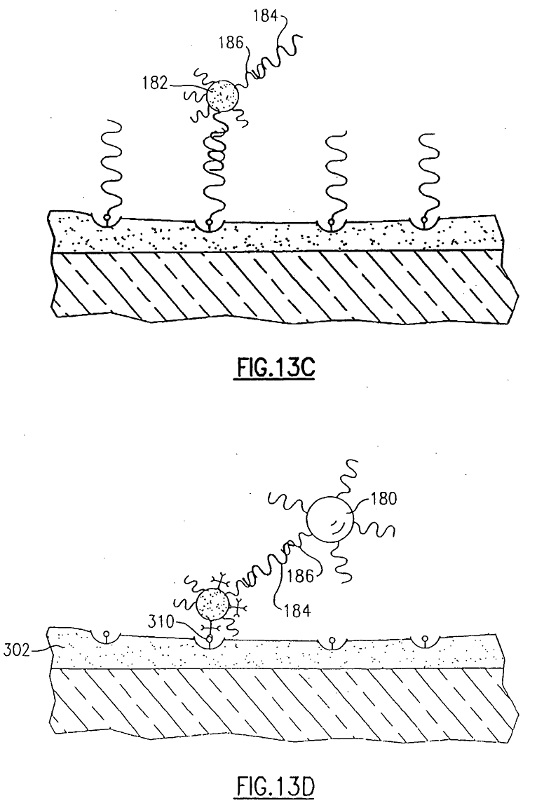

FIGS. 13A-13C are detailed partial cross sectional views showing a capture layer 302 over a substrate 304 of an optical disc. A capture agent 306 attached to the capture layer 302 by use of an amino group 308 which is made an integral part of the capture agent 306. Capture agent 306 is situated within a capture field. The bond between the amino group 308 and the capture agent 306, and the amino group 308 and the capture layer 302 is sufficient so that the capture agent 306 remains attached to the capture layer 302 within the capture field when the disc is rotated. The preferred amino group 308 is NH₂. A thiol group can be employed in place of the amino group 308.

Reporter bead 182 from dual bead complex 190 binds to capture agent 306 in the capture field. In this embodiment, a separate capture agent is not required and the BSA-biotin forms capture layer 302 and serves as a capture agent, and the Streptavidin-coated reporter bead 182 binds directly to the affinity agent 310 (biotin) in capture layer 302 without the need for a linking transport probe and amino group.

In the case of DNA, the capture agent can be single stranded or partially double stranded near the attachment point to the capture layer. One embodiment of the capture agent includes double stranded DNA at the capture layer because the double stranded DNA has been found to more effectively project erectly or upwardly from the capture layer as compared to single stranded DNA in some instances. In the case of a partially double-stranded capture agent, an extension of single stranded DNA is employed so that hybridization can occur with a target DNA.

Each reporter bead 182 is pretreated with designated signal probes 188 conjugated onto the bead preferably by using a carboxyl group.

Reporter bead 182 can be captured in the capture field 148 via BSA-biotin/streptavidin interactions.

The disc can be rotated to move unbound beads away from the capture field. In this embodiment, unbound magnetic capture beads (see FIGS. 11 and 12) will be moved away from the capture field through the rotation, thereby leaving just bound dual bead complex structures with detectable reporter beads.

FIG. 13C illustrates an alternative embodiment which includes an additional step over that shown in FIGS. 13A-13B. In this embodiment, the disc is rotated to create sufficient centrifugal force to break capture beads 180 away from dual bead complex 190 based on the differential size and/or mass of the bead. Although there can be a shift in the rotational speed of the disc, the speed is such that reporter bead 182 remains anchored to capture layer 302. In either case, reporter beads 182 are maintained within the capture field.

Referring to FIG. 13D, the reporter bead can have biotinylated signal probes or the reporter beads can be coated with an affinity agent such as streptavidin, and the capture area can include BSA-biotin for capturing capture beads coated with streptavidin, or the streptavidin binds to biotinylated transport probes on the capture beads. As dual bead complex 190 flows towards capture area 302 and is in sufficient proximity thereto, binding occurs between the dual bead complex 190 and the affinity agent 310 on the surface of capture field 302.

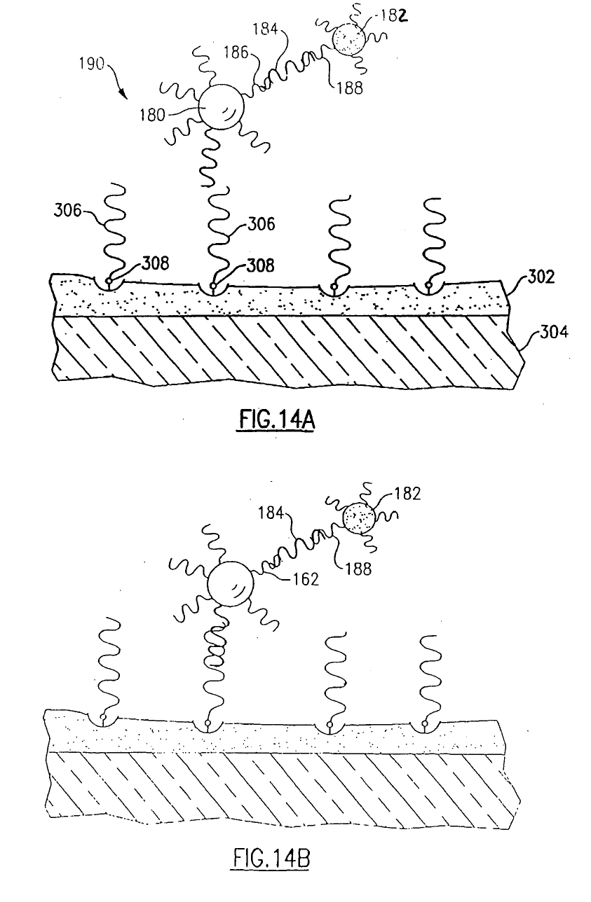

FIGS. 14A and 14B are detailed partial cross sectional views showing capture layer 302 and substrate 304 in another embodiment. Capture agent 306 is attached to capture layer 302 by use of an amino group 308 which is made an integral part of the capture agent. The bond between amino group 308 and capture agent 306, and amino group 308 and capture layer 302 is sufficient so that the capture agent remains attached to capture layer 302 within the capture field when the disc is rotated. The preferred amino group 308 is NH₂. A thiol group can alternatively be employed in place of the amino group 308.

As described in conjunction with FIG. 13D, the capture layer can include BSA-biotin for capturing reporter beads coated with streptavidin, or it can have streptavidin for capturing biotinylated signal probes on reporter beads. In this embodiment, capture bead 180 anchors the dual bead complex 190 to capture agent 306, preferably via BSA-biotin and streptavidin interactions. Alternatively, the capture layer can be composed of streptavidin and can bind to biotinylated capture beads.

In this embodiment, capture beads not part of a dual bead complex and remaining in the isolate will also be captured, but then will be undetectable because reporter beads are not attached. With the use of fluorescent reporter beads, or in an embodiment where a yes/no answer is sufficient, having unbound capture beads attach can be acceptable.

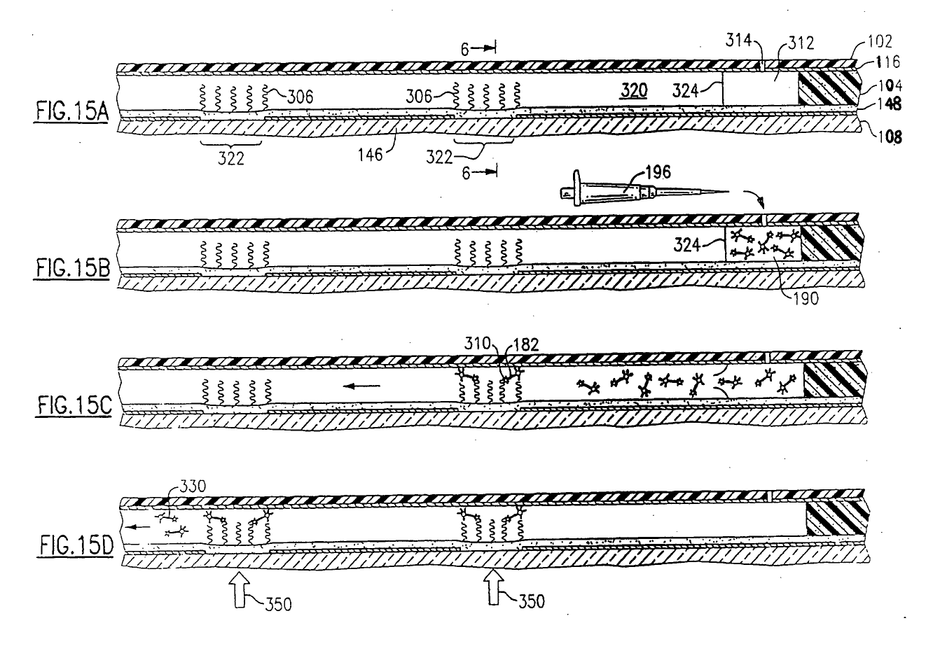

FIGS. 15A-15D show the capture areas set out in FIGS. 13A-13C and FIGS. 14A-14B in the context of a disc, using as an input the solution created according to methods such as those shown in FIGS. 11 and 12.

FIG. 15A shows a loading chamber 180, accessible through a port 314, and leading to a flow channel 320. Flow channel 320 is pre-loaded with capture agents 306 situated in clusters in capture fields 322. Each of the clusters of capture agents 306 are situated within a respective capture field 322. Each capture fields 322 can have one type of capture agent or multiple types of capture agents, and separate capture fields can have one and the same type of capture agent or multiple different capture agents in multiple capture fields.

In FIG. 15B, a pipette 196 is loaded with a test sample of DNA that has been sequestered in the dual bead complex 190. The dual bead assay is injected into flow channel 320 through inlet port 314. As flow channel 320 is further filled with the dual bead assay from pipette 196, the dual bead complex 190 begins to move down flow channel 320 as the disc is rotated. The loading chamber 180 can include a break-away retaining wall 324 so that complex 190 moves down the flow channel at one time.

In this embodiment, binding agents on reporter beads 182 bind to the affinity agents 310. In this manner, reporter beads 182 are retained within capture fields 322 via BSA-biotin/Streptavidin interactions. Binding can be further facilitated by rotating the disc so that the dual bead complex 190 can slowly move or tumble down the flow channel. Slow movement allows ample time for additional hybridization. After hybridization, the disc can be rotated further at the same speed or faster to clear capture fields 322 of any unattached dual bead complex 330.

An incident light beam 350 can then be scanned through capture fields 322 to determine the presence of reporters as illustrated in FIG. 15D. In the event no target DNA is present in the test sample, there are no dual bead complex structures formed in the assay, but a small amount of background signals are detected in the capture fields from unspecific binding. In this case, when the interrogation beam 350 is directed into the capture fields 148, a negative reading results thereby indicating that no target DNA was present in the sample.

The speed, direction, and stages of rotation, such as one speed for one period followed by another speed for another period, can all be encoded in the operational information on the disc.

Summary of Dual Bead Assays for Target Nucleic Acids

In one specific embodiment of the present invention, the target molecules are nucleic acids (e.g., DNA, RNA and chemical analogs thereof) and the dual bead assay employs approximately 3 micron magnetic capture beads and approximately 2.1 micron fluorescent reporter beads. These beads are coated with transport probes and signal probes respectively. The transport probes and signal probes are complementary to the target sequence but not to each other. The capture beads are mixed with varying quantities of target nucleic acid. Unbound target nucleic acid is removed from the solution by magnetic concentration of the magnetic beads. Fluorescent reporter beads are then allowed to bind to the captured target DNA. Unbound reporter beads are removed by magnetic concentration of the magnetic beads. Thus, the magnetic capture beads bind to fluorescent reporter beads, only in the presence of the target sequence, resulting in a dual bead assay.

The transport probes and signal probes are covalently conjugated onto carboxylated capture beads and reporter beads via EDC conjugation. Attaching double stranded probes to the beads prior to conjugation reduces the non-covalent attachment of probes to beads significantly. By using appropriate bead type and conjugation conditions, the covalent conjugation efficiency could be as high as 95%.

The use of magnetic beads in the capture of target nucleic acid speeds up the washing steps and facilitates the separation steps between bound and unbound target sequences or antigens/antibodies significantly. Furthermore, when the target concentration is limiting, each target agent will hybridize to one reporter bead. One target agent is not detectable by any existing technologies. In the dual bead assay, detection of the target is assisted by the use of approximately 2.1 micron reporter beads. Reporter beads can be easily detected and quantified by various methods. Therefore, the dual bead assay increases the sensitivity of the target capture.

Different methods can be employed for immobilizing capture reagents in the capture field. One method includes using BSA-biotin molecules to capture streptavidin or neutravidin coated reporter beads. A second method includes using a nucleic acid sequence complementary to the signal probes to capture the reporter beads. In the first method, the disc surface is coated with a layer of polystyrene, and then BSA-biotin are spotted onto capture fields located on the disc. In the second method, the capturing sequence is modified at the end with an amino group, and the disc surface is coated with maleic anhydride polystyrene. The amino group on the probe binds covalently to the maleic anhydride, thereby attaching the nucleic acid transport probe to the disc in the capture field. Unbound capture reagents are washed off.

After this binding, the channel is formed by affixing the adhesive layer and a cover or cap layer (see FIGS. 5 and 6).

The dual bead assay suspension is then loaded into the channels via the port such that the whole channel is filled with the sample. The ports are sealed and the disc is rotated in the optical disc drive. The reporter beads (with or without the attaching magnetic capture beads) can be captured within the capture field. During spinning, free magnetic capture beads will be spun off to the bottom of the channel. Alternatively, all magnetic capture beads can be spun off to the bottom of the channel, and only the reporter beads remain bound to the capturing field.

Detection

The number of reporter beads bound in the capture field can be detected in a yes/no manner, and/or can be quantified by the optical disc drive/reader.

The test results of any of the test methods described above can be readily displayed on monitor 14 (FIG. 1). The disc according to the present invention preferably includes encoded software that is read to control the controller, the processor, and the analyzer as shown in FIG. 4. This interactive software is implemented to facilitate the methods described herein and the display of results.

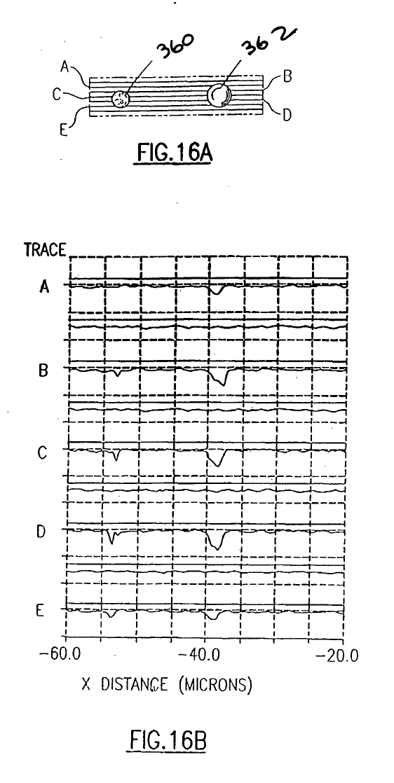

Referring to the graph of FIGS. 16A and 16B, a number of traces can be made with the incident beam to produce two different signatures. These graphs represent detected reflected light. As shown, the signatures for a 2.1 micron reporter bead 360 are sufficiently different from those for a 3 micron capture bead 362 such that the two different types of beads can be detected and discriminated. A sufficient change in detected light is referred to as an event.

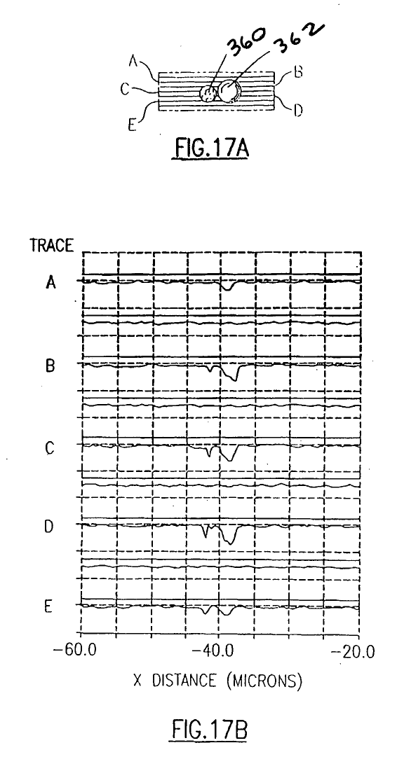

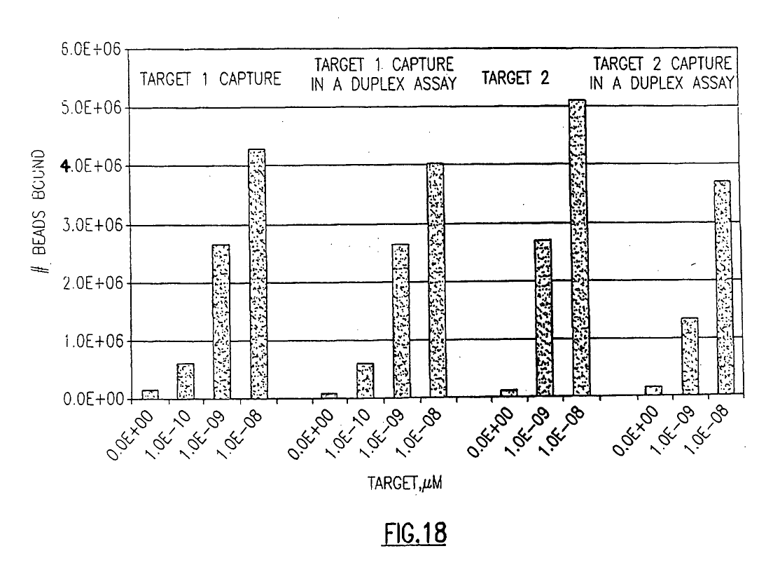

FIGS. 17A and 17B show scan lines and graphs indicating the detection of a reporter bead 360 and a capture bead 362 bound together into an oblong shape of a dual bead complex.

Alternatively, other detection methods can be used. For example, reporter beads can be used that fluoresce at a known wavelength. The disc drive is set at the desired wavelength or is controllable to provide light at that wavelength. The bead then emits light at a different wavelength. The emitted light can be detected by a detector that is dedicated to that wavelength or is controllable to detect that wavelength, e.g., though the use of filtering prior to the detector. The wavelength that is used for the light source and the emitted light can be encoded on the disc or provided by the user. The user need not know specifically the wavelength, but a user interface can allow a user to select from among different types of tests, thereby reducing the need for technical knowledge by the user. Detection methods are described, for example, in an application entitled “Disc Drive System and Methods for Use with Bio-Discs” filed Nov. 9, 2001, as Ser. No. 10/008,156, which is expressly incorporated by reference; and also Provisional Application Nos. 60/270,095 filed Feb. 20, 2001 and 60/292,108, filed May 18, 2001.

Sensitivity

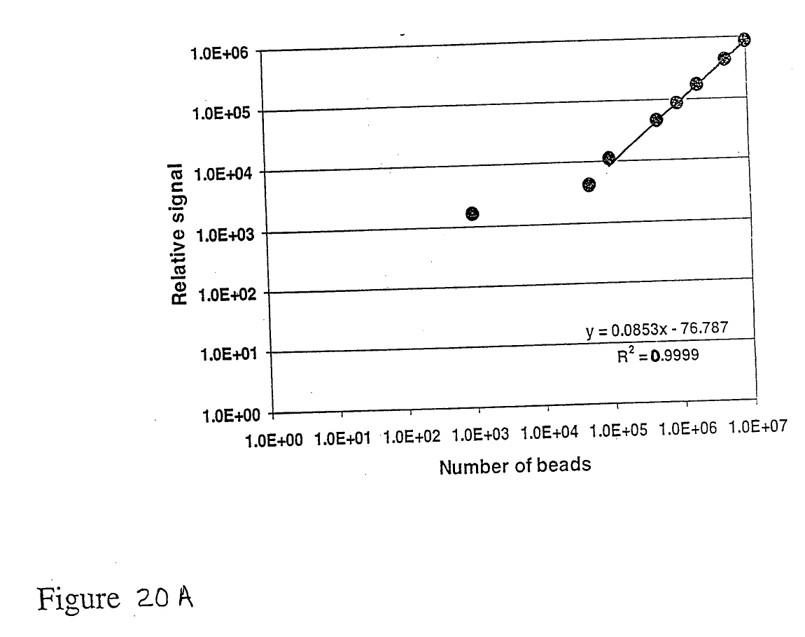

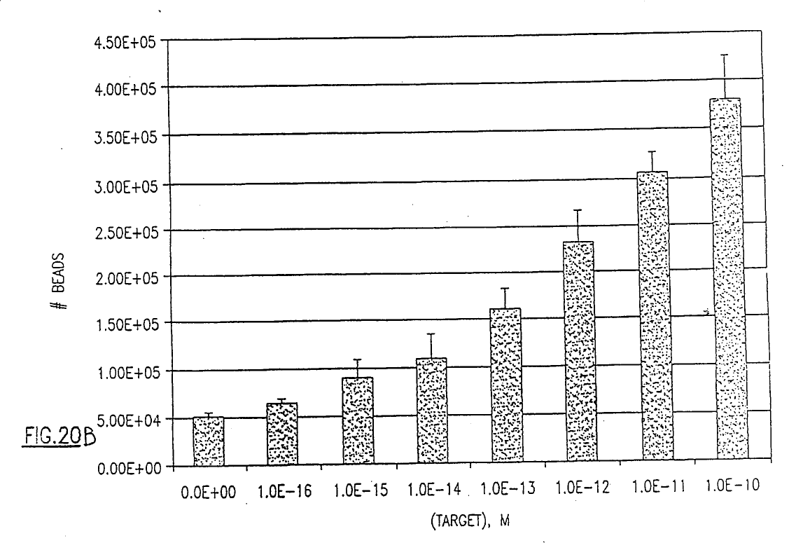

The sensitivity of any assay depends on the sensitivity of the assay itself and on the sensitivity of the detection system. Referring to FIGS. 20A-20C, various studies were done to examine the sensitivity of the dual bead assay using conventional detection methods, e.g., a fluorimeter, and biodisc detection. FIG. 20A presents a standard curve demonstrating that the sensitivity of a fluorimeter is approximately 100 beads in a fluorescent dual bead assay. In FIG. 20B, a graph showing mole concentration versus number of detected beads shows that even at 10E−16 Molar (moles/liter), a sufficient number of beads over zero concentration can be detected to sense the presence of the target. With a sensitivity of 10E−16 Molar, a dual bead assay represents a very sensitive detection method for DNA that does not require DNA amplification (such as through PCR) and can be used to detect even a single bead.

In contrast to conventional detection methods, the use of a biodisc coupled with a CD-reader (FIG. 1) improves the sensitivity of detection. For example, while detection with a fluorimeter is limited to approximately 1000 beads (FIG. 20A), use of a biodisc coupled with CD-reader enables the user to detect a single bead with the interrogation beam (FIG. 20C). Thus, the bioassay system provided herein improves the sensitivity of dual bead assays significantly.

Multiplexing Assays

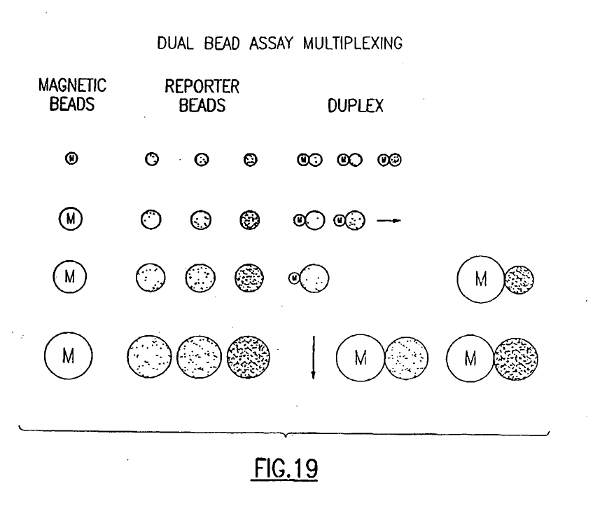

The use of a dual bead assay in the capture of targets allows for the use of multiplexing assays. Referring to FIG. 19, by combining different sizes of magnetic beads and different types and sizes of reporter beads, different target agents can be detected simultaneously. As indicated in FIG. 19, four sizes of magnetic capture beads, and four sizes of three types of reporter beads produce up to 48 different types of dual bead complex. In a multiplexing assay, probes specific to different targets are thus conjugated to capture beads and reporter beads having different physical and/or optical properties, such as fluorescence at different wavelengths, to allow for the detection of different target agents simultaneously from the same biological sample in the same assay. As indicated above, small differences in size can be detected by detecting reflected or transmitted light.

Multiple dual bead complex structures to capture different target agents can be carried out on or off the disc. If off the disc, the dual bead suspension is loaded into a port on the disc. The port is sealed and the disc is rotated in the disc reader. During spinning, free (unbound) beads are spun off to a periphery of the disc. The reporter beads detecting various target agents are thus localized in capture fields. In this manner, the presence of a specific target agent can be detected, and the amount of a specific target agent can be quantified by the disc reader.

Referring to FIG. 18, a graph (generated with data from a fluorimeter) demonstrates the detection of two targets and shows that for different concentrations of target in a solution, large numbers of beads can be detected.

Creation of Dual Bead Complex on the Disc

The exemplary methods of FIGS. 11 and 12 are for preparation of dual bead complexes outside of the disc for later injection into the disc as shown in FIGS. 15A-15D.

Alternatively, the dual bead complex can be formed in whole on the disc with just a sample injected, or in part with the sample and some beads injected. In one embodiment, the beads and sample are added to the disc at the same time, or nearly the same time. Alternatively, the beads with the probes can be pre-loaded on the disc for future use with a sample so that only a sample needs to be added. This latter embodiment should produce the easiest to operate version of a dual bead complex disc.

The beads would typically have a long shelf life, with less shelf life for the probes. The probes can be dried or lyophilized (freeze dried) to extend the period during which the probes can remain in the disc. With the probes dried, the sample essentially reconstitutes the probes and then mixes with the beads to produce dual bead complex structures can be performed.

In either case, the basic process for on disc processing includes:
(1) inserting the sample into a disc with beads with probes;
(2) causing the sample and the beads to mix on the disc;
(3) isolating, such as by applying a magnetic field, to hold the dual bead complex and move the non-held beads away, such as to a region referred to here as a waste chamber; and
(4) directing the dual bead complexes (and any other material not moved to the waste chamber) to the capture fields.

The detection process can be the same as one of those described above, such as by event detection or fluorimetry.

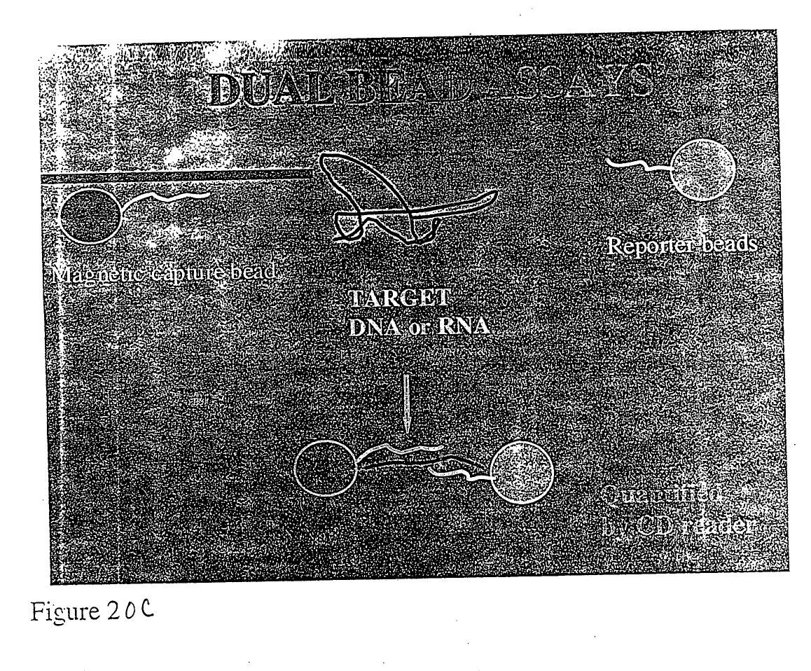

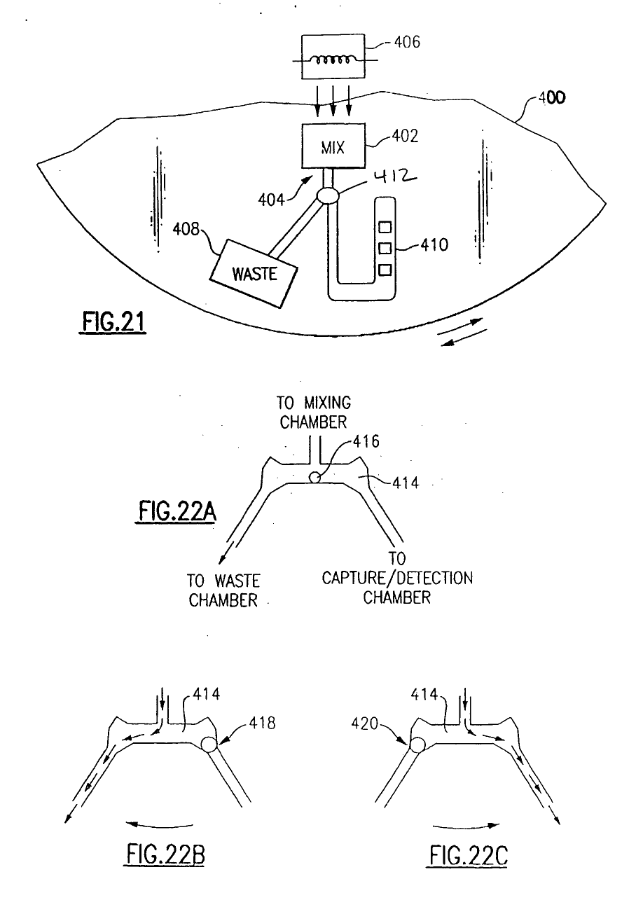

Referring to FIG. 21, a general representation of a disc according to this aspect of the present invention and a method corresponding generally to the one-step method of FIG. 11 is shown. The sample and beads can be added at one time or successively but closely in time, or the beads can be pre-loaded into a portion of the disc. These materials can be provided to a mixing chamber 402 that can have a breakaway wall (see FIG. 15A) or an exit port 404 that has capillary forces that hold in the mixture. Mixing the sample and beads on the disc would be accomplished through rotation at a rate insufficient to cause the wall to break or the capillary forces to be overcome.

The disc can be rotated in one direction, or it can be rotated alternately in opposite directions to agitate the material in a mixing chamber. The mixing chamber is preferably sufficiently large so that circulation and mixing is possible. The mixing can be continuous or intermittent.

Next, in the case of the capture beads being magnetic, a magnetic field from a source 406 can be applied over mixing chamber 402 to hold the dual bead complexes and unbound magnetic beads in place while material without magnetic beads is allowed to flow away to a waste chamber 408 or to be trapped in a side area of the disc. At this stage, only magnetic capture beads, unbound or as part of a dual bead complex, remain. The magnetic field is released, and the dual bead complex with the magnetic beads is directed to capture and detection chamber 410.

The process of directing non-magnetic beads to waste chamber 408 and then magnetic beads to detection chamber 410 can be accomplished through the microfluidic construction and/or fluidic components. A valve 412 or some other directing arrangement can be used to direct the sample and non-magnetic beads to waste chamber 408 and then to detection chamber 410. A number of embodiments for rotationally dependent flow can be used.

FIG. 22A shows one embodiment of a rotationally directionally dependent valve arrangement that is directionally dependent and uses a movable component for a valve. The mixing chamber leads to an intermediate chamber 414 that has a movable component, such as a ball 416. In the non-rotated state, the ball may be kept in a slight recessed portion, or chamber 414 may have a gradual V-shaped tapering in the circumferential direction to keep the ball centered when there is no rotation.

Referring to FIGS. 22B and 22C in addition to FIGS. 21 and 22A, when the disc is rotated clockwise (FIG. 22B), ball 416 moves to a valve seat 418 to block passage to detection chamber 410 and to allow flow to waste chamber 408. When the disc is rotated counter-clockwise (FIG. 22C), ball 416 moves to a valve seat 420 to block a passage to waste chamber 408 and to allow flow to detection chamber 410.

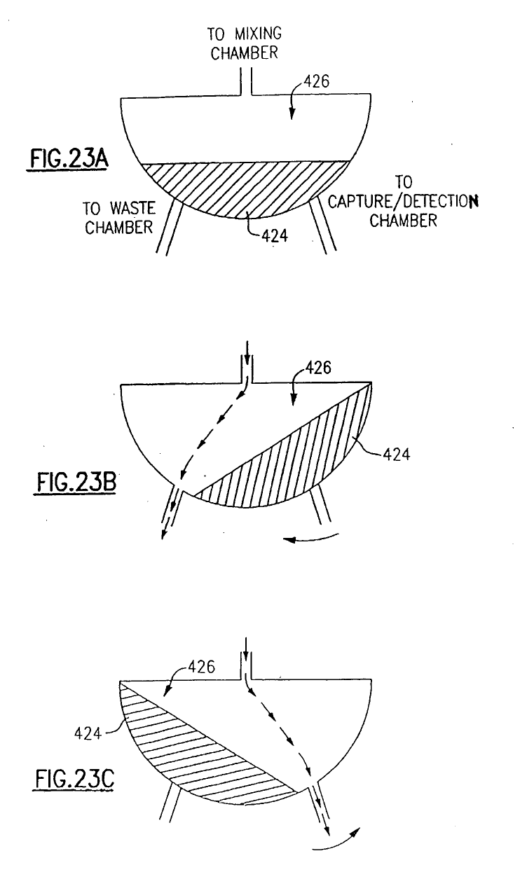

FIGS. 23A-23C show a variation of the prior embodiment in which the ball is replaced by a wedge 424 that moves one way or the other in response to acceleration of the disc. The wedge can have a circular outer shape that conforms to the shape of an intermediate chamber 426. The wedge is preferably made of a heavy dense material relative to chamber 426 to avoid sticking. A coating can be used to promote sliding of the wedge relative to the chamber.

When the disc is initially rotated clockwise (FIG. 23B), the angular acceleration causes wedge 424 to move to block a passage to detection chamber 410 and to allow flow to waste chamber 408. When the disc is initially rotated counter-clockwise (FIG. 23C), the angular acceleration causes wedge 424 moves to block a passage to waste chamber 408 and allow flow to detection chamber 410. During constant rotation after the acceleration, wedge 424 remains in place blocking the appropriate passage.

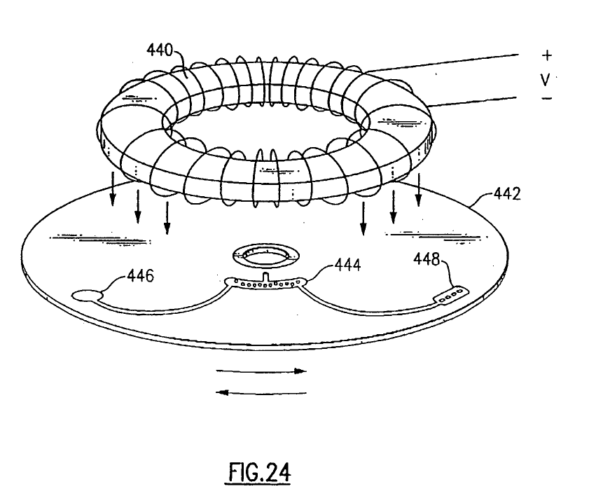

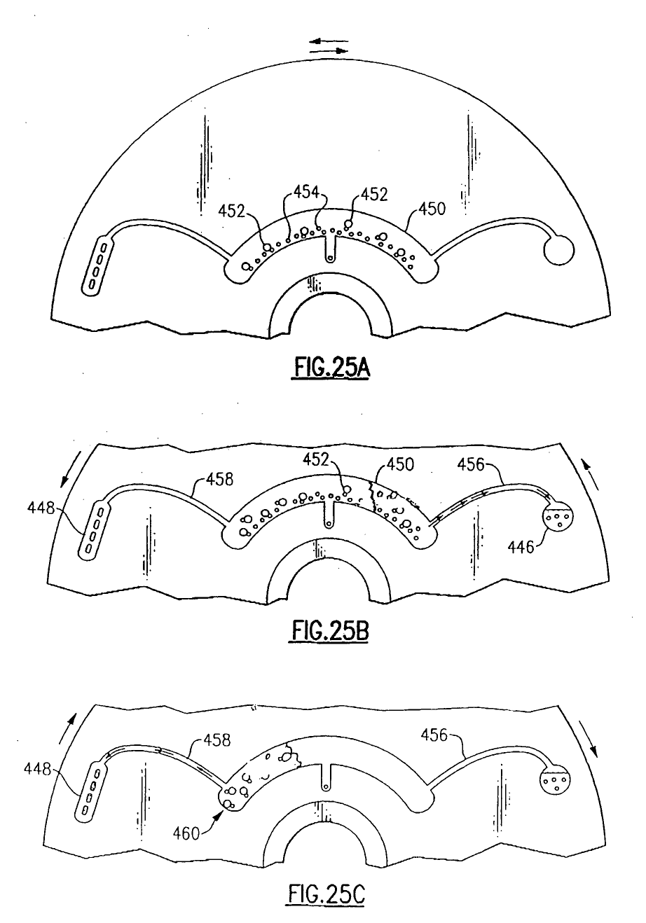

FIGS. 24 and 25A-25C show another embodiment, demonstrating that control over the flow can be performed without moving parts but through the configuration of passages. In FIG. 24, a disc 442 has a mixing chamber 444, a waste chamber 446, and a detection chamber 448. An annular electromagnet 440 is positioned over disc 442 and has a radius such that as disc 442 rotates, electromagnet 440 remains over mixing chamber 444, and is radially spaced from chambers 446 and 448.

Referring to FIGS. 25A-25C, in the non-rotated state (FIG. 25A), a mixing chamber 450 shaped as an annular sector holds a sample with dual bead complexes 452 and various unbound magnetic capture beads and reporter beads 454. The electromagnet is turned on and the disc is rotated counter-clockwise (FIG. 25B), or it can be agitated at a lower rpm, such as 1× or 3×. Dual bead complexes 452 remain in mixing chamber 450 while the liquid sample moves in response to angular acceleration to a rotationally trailing end of mixing chamber 450. The disc is rotated with sufficient speed to overcome capillary forces to allow the non-held part of the sample to move through a passage 456 to waste chamber 446.

Next, the magnet is turned off and the disc is rotated counter-clockwise (FIG. 25C). Dual bead complexes 452 and other magnetic beads move to now-trailing end 460 in response to angular acceleration and then through a passage 458 to detection chamber 448. The liquid cannot move down passage 456 at this stage because of the configuration. This embodiment thus illustrates directionally dependent flow as well as rotational speed dependent flow.

In this embodiment and others in which a fluidic circuit is formed in a region of the disc, plurality of regions can be formed and distributed about the disc, for example, in a regular manner to promote balance. Furthermore, as discussed above, instructions for controlling the rotation can be provided on the disc. Accordingly, by reading the disc, the disc drive can have instructions to rotate for a particular period of time at a particular speed, stop for some period of time, and rotate in the opposite direction for another period of time. In addition, the encoded information can include directions such as the power and wavelength of the light source, particularly if fluorescence is used, and other such parameters.

Other embodiments for controlling flow can be used, in some cases by using the electromagnet, and in some cases by using movable parts (in addition to the disc itself, such as a ball or wedge) or without additional movable parts. Capillary forces can be used to make the flow tend to go to the detection chamber unless the passage to the detection chamber is blocked and the material is thus directed to the waste chamber. This blockage can occur in response to the magnetic field, such as with a ball or movable wall that moves across the passage that leads to the detection chamber.

In yet another embodiment, a passage can have a material or configuration that can seal or dissolve either under influence from a laser in a disc drive, or a catalyst provided onto the disc, such as in the sample. For example, a gel may solidify in the presence of a material over time, in which case the time to close can be set sufficiently long to allow the unbound capture beads to flow to a waste chamber before the passage to the waste chamber closes. Alternatively, the passage to the waste chamber can be open while the passage to the detection chamber is closed. After the unbound beads are directed to the waste chamber, the passage to the direction chamber is opened by energy introduced from the laser to allow flow to the detection chamber.

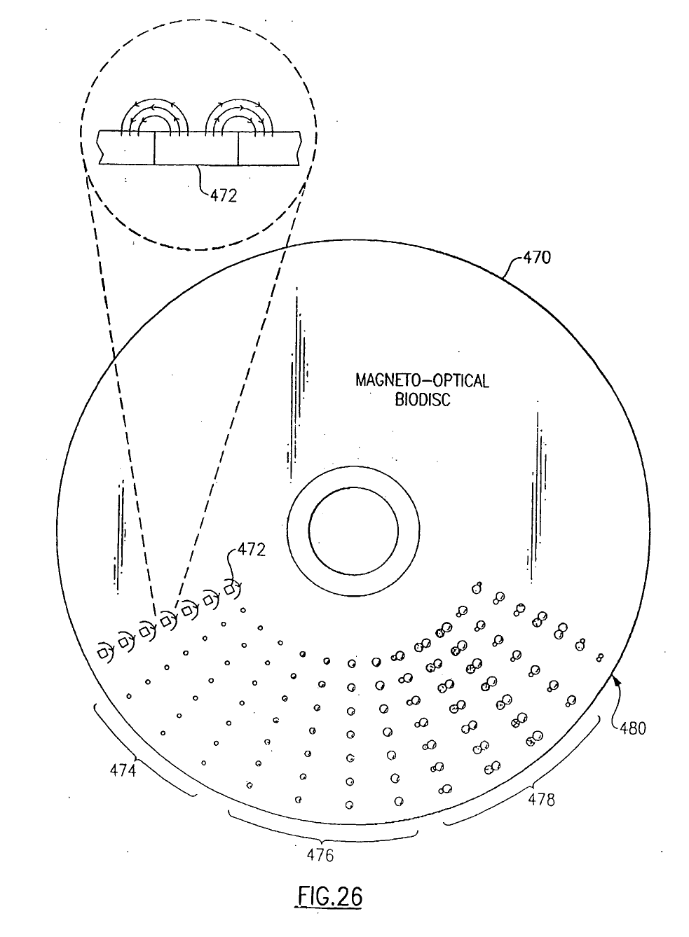

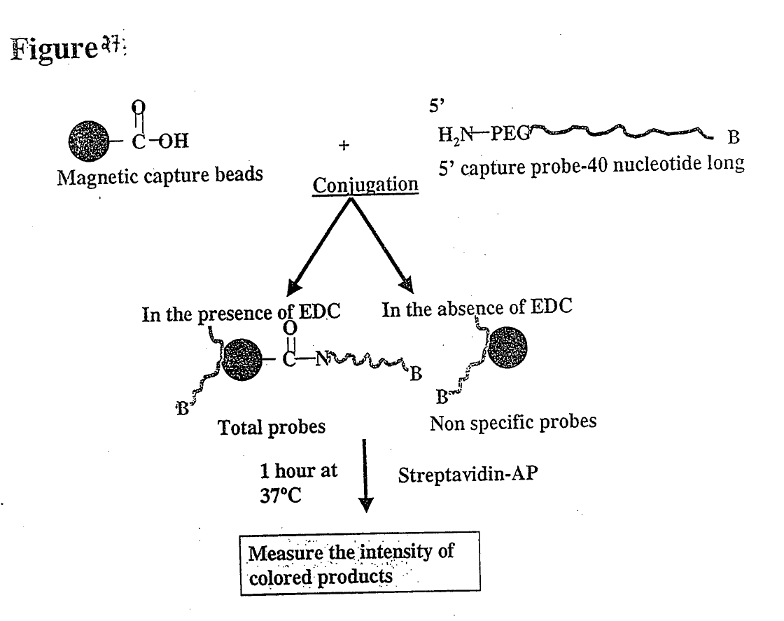

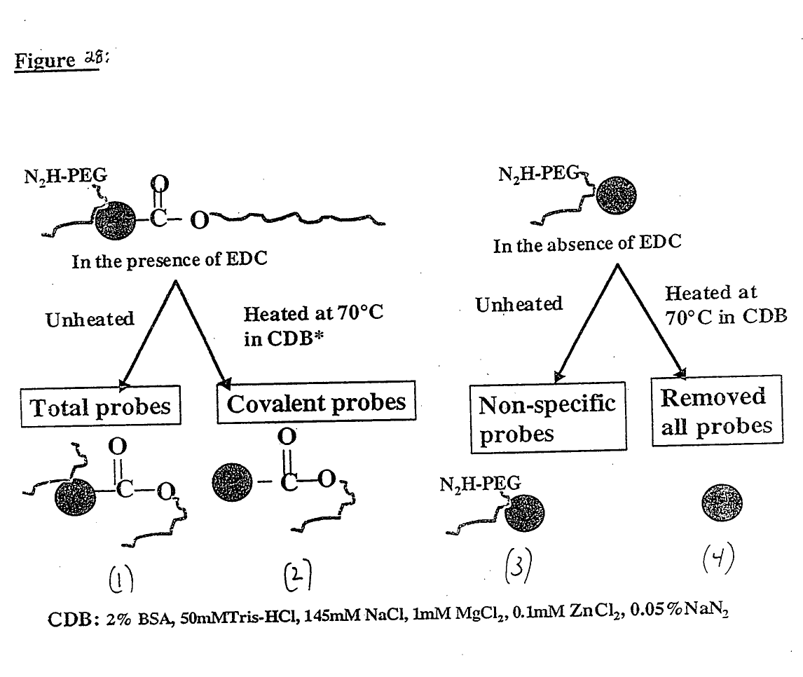

Referring to FIG. 26, another embodiment of a disc 470 for use with dual bead complexes is shown. In this case, a disc, such as one used with a magneto-optical drive, has regions that can be written and erased with a magnetic head and read with an optical reader. A magneto-optical disc drive, for example, can write in regions as small as 1 micron by 1 micron square, shown as squares 472. As indicated in the close-up section, magnetic field lines are shown with respect to adjacent regions.

The ability to write to small areas in a highly controllable manner to make them magnetic allows capture areas to be created in desired locations. These magnetic capture areas can be formed in any desired configuration or location in one chamber or in multiple chambers. These areas capture and hold magnetic beads when applied over the disc. The domains can be erased if desired, thereby allowing them to be made non-magnetic and allowing the beads to be released.

In one configuration, a set of three radially oriented columns 474 are shown with no beads attached to the squares in the columns. A set of four columns 476 is shown with individual magnetic beads magnetically attached to the squares in the columns. A set of four columns 478 is shown with dual bead complexes attached to the squares in the columns, with different columns having different sets of magnetic beads (note that some beads are larger than others). Final column 480 is shown with different dual bead complexes attached at different squares.

In a method for use with such a disc, the write head can be used to create magnetic squares, and then the sample can be flowed over that area to capture magnetic beads in the sample. Then an area with a new set of squares can be made magnetic and the sample provided to that area to bind to the disc at the squares. If desired, the regions can be erased, thereby making them non-magnetic and allowing captured beads to be released. Thus this system allows one or more highly controllable capture areas to be created. The spaced apart squares can also make detection easier because of the spacing and the known locations.

As described above, a sample can be provided to a chamber on a disc, but naturally a sample could be provided to multiple chambers that have sets of beads different from each other. Alternatively, a series of chambers can be created such that a sample can be moved through rotational motion from one chamber to the next, and different tests can then be performed.

With such a disc, a large number of tests can be performed at one time and can be performed interactively, such that when a test is performed and a result is obtained, the system can be instructed to create a new set of magnetic regions for capturing the dual bead complex. Regions can be created one at a time or in large groups, and can be performed in successive chambers that have different pre-loaded beads. Other processing advantages can be obtained with a disc that has writable magnetic regions. For example, the “capture agent” is essentially the magnetic field created by the region on the disc and therefore there is no need to add an additional capture agent. By using rotation, the amount of buffering and washing can be reduced or possibly even eliminated.

Instructions for controlling the locations for regions written or erased on the disc, and other information such as rotational speeds, stages of rotation, waiting periods, wavelength of the light source, and other parameters can be encoded on and then read from the disc itself.

Successful conjugation of a probe(s) to a solid phase, e.g., a bead or a biodisc, is an important step for the dual bead assays of the invention. In certain embodiments of the invention, probes are attached covalently to the beads. Efficiency of the covalent conjugation depends on the type of bead utilized and the specific conjugation method employed.

As illustrated in FIG. 27, a systematic method to evaluate the use of a solid phase for probe conjugation is presented. The methodology identifies covalent linkages that improve specificity of a dual bead assay. This approach can be used to evaluate treatment of solid phase (i.e., coating of a solid surface such as the surface of a bead or a surface on a biodisc) to see whether the treatment improves the solid phase conjugation efficiency. As a first step, a probe(s) is tagged with an appropriate molecule for detection and measurement of the amount of probe bound at a later time. By way of non-limiting example, a biotin moiety (B) can be attached at the 3′ end of a DNA probe. Next, the probe(s) is conjugated in the presence or absence of a cross-linking agent, e.g., EDC (1-Ethyl 3-3 dimethylaminopropyl carbodiimide-HCl). In the presence of a cross-linking agent, a probe(s) will be conjugated both covalently and non-covalently. Alternatively, in the absence of the cross-linking agent, a probe(s) will only be absorbed to the bead non-covalently. After the appropriate washing steps are performed, a detection agent is added that binds specifically to the biotin molecule previously tagged to the probe. For example, streptavidin-alkaline phosphatase (S-AP) is added to the probe-bound beads, and the S-AP binds specifically to the biotinylated probe(s). Next, alkaline phosphatase substrate is added to the sample. This substrate develops color upon loss of a phosphate group, and the intensity of the color correlates with the amount of probes bound to the beads. After an appropriate incubation period, the solution is isolated and the optical density of the solution at an appropriate wavelength is determined with a spectrophotometer or microtiter plate reader.

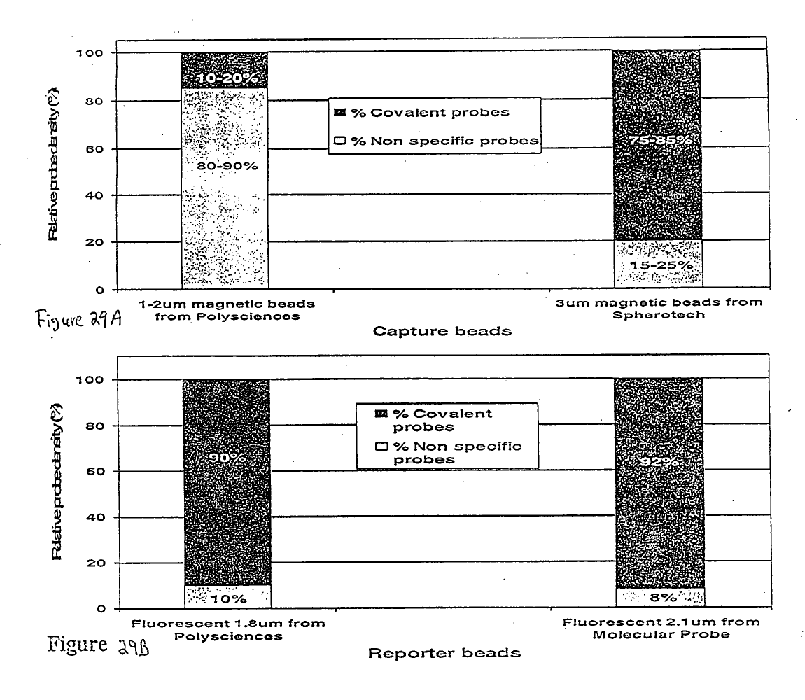

Referring to FIG. 28, the amount of probe covalently bound to the solid surface may be determined by determining the amount of probe that binds to the solid phase covalently and non-covalently, i.e., non-specifically, in the presence and absence of a crosslinking agent (e.g., EDC). The percentage of non-covalently bound probe can be determined according to the formula 100%*N/T, and the percentage of covalently bound probe can be determined by the formula 100%*(T−N)/T, wherein “T” represents the total amount of signal obtained in the presence of a cross-linking agent (i.e., the total amount of covalently and noncovalently bound probe) and “N” represents the total amount of signal obtained when no crosslinking agent is used. Alternatively, the amount of probe(s) conjugated covalently can be obtained directly if all non-covalently bound probe is removed prior to the addition of the S-AP. This can be conveniently achieved by heating the beads to 70° C. prior to the step of adding the S-AP. If the percentage of non-covalently bound probe is less than 20%, the beads being tested can be used as solid phase for covalent conjugation. Results of an application of this methodology are presented in FIGS. 29A and 29B (see Example 3 for details).

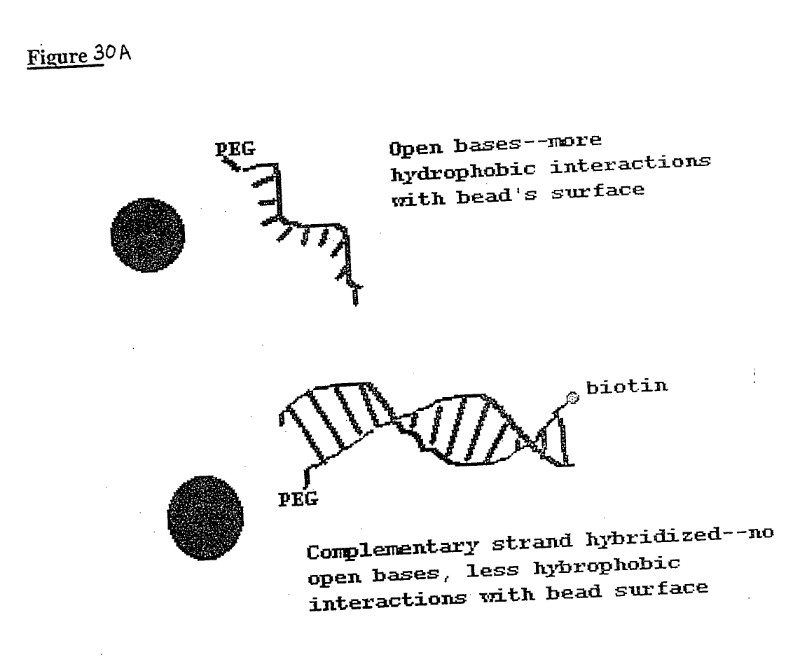

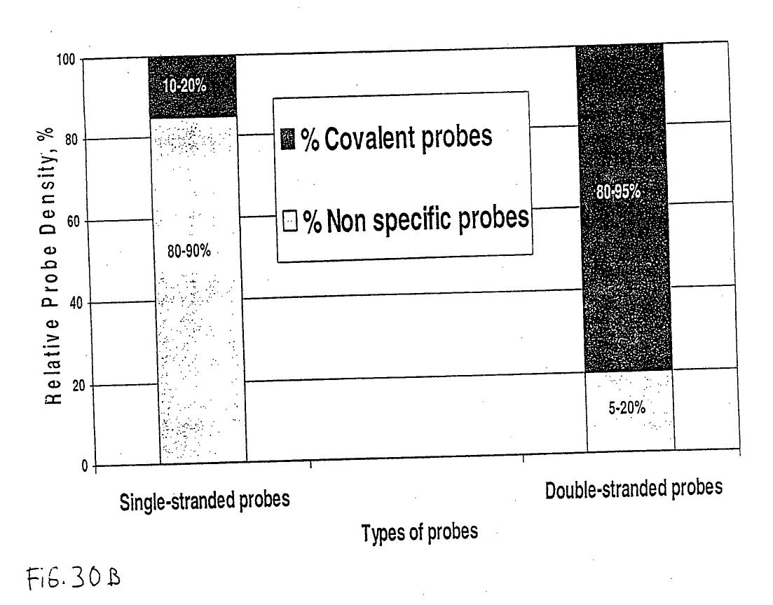

Various embodiments of the invention utilize nucleic acid molecules as probes. FIG. 30A shows the structural differences between single stranded and double stranded DNA in order to illustrate how the single stranded DNA can more readily bind non-covalently to a solid phase. Single-stranded DNA has hydrophobic base side chains that can readily absorb to a solid phase non-covalently. In contrast, with double-stranded DNA hydrophobic base interaction with a solid phase does not generally occur and non-covalent or non-specific binding is more limited in comparison to a single-stranded DNA molecule (FIG. 30B). Thus, in various embodiments of the invention, double stranded DNA can be utilized in place of single-stranded DNA, thereby limiting DNA binding to a solid phase by covalent linkage. After crosslinking of double-stranded DNA to the solid phase, single stranded probes for target capture can be obtained by heating the sample to 70° C. in the appropriate buffer. Under these condition, the strands of the double stranded DNA are separated, and only single stranded DNA is covalently attached to the beads.

In various embodiments of the invention, a probe(s) can be attached to a solid phase by way of a linker molecule. The use of a linker molecule makes the probe longer and more rigid. These two properties increase the accessibility of the probe(s), and, therefore, maximize the efficiency of target capture and the sensitivity of the dual bead assay. As known to those skilled in the art, various linker molecules can be used that satisfy the criteria described herein. By way of non-limiting example, bovine serum albumin (BSA) or polyethylene glycol (PEG) can be used as linker molecules. In certain embodiments of the invention, the linker can be a series of 3 to 10 PEG molecules that are attached to the 5′ end of a DNA probe(s) covalently.

In various embodiments of the invention, heat treatment can be used to selectively remove non-covalently bound probe(s) from a solid phase. Such as strategy is useful when, for example, despite all optimizations with respect to the type of the solid phase, treatment of the solid phase, and the use of double stranded DNA, non-covalent binding to the solid phase is still problematic. The conditions for the heat treatment have been optimized; the optimal buffer consists of: 2% BSA, 50 mM Tris-HCl, 145 mM NaCl, 1 mM MgCl2, 0.1 mM ZnCl2. The treatment is done at a temperature less than or equal to 70° C., since at higher temperatures, the magnetic beads can lose their magnetic properties.

In other embodiments of the invention, the methodology presented herein to determine optimal conditions to obtain covalent linkages that improve specificity of a dual bead assay can be applied to a disc surface that is used as a solid phase. Similarly, the invention provides in other embodiments analogous to those described herein above to evaluate solid surfaces for protein binding. For example, such an application would be useful where the probe utilized is an antigen or antibody.

### EXPERIMENTAL DETAILS

While this invention has been described in detail with reference to certain examples and further illustrations of the invention are in the experimental details section which follows, it should be appreciated that the present invention is not limited to the precise examples. Rather, in view of the present disclosure, many modifications and variations would present themselves to those of skill in the art without departing from the scope and spirit of this invention. The examples provided are set forth to aid in an understanding of the invention but are not intended to, and should not be construed to, limit in any way the invention as set forth.

### Example 1

1. Dual Bead Assay

In this example, the dual bead assay is carried out to detect the gene sequence DYS that is present in male but not female. The assay is comprised of 3 micron magnetic capture beads (Spherotech, Libertyville, Ill.) coated with covalently attached capture probes (Biosource, CA); 2.1 micron fluorescent reporter beads (Molecular Probes, Eugene, Oreg.) coated with a covalently attached sequence (Biosource, CA) specific for the DYS gene, and target DNA molecules containing DYS sequences. The target DNA is a synthetic 80 oligonucleotide sequence. The capture probes and reporter probes are 40 nucleotides in length and are complementary to the DYS sequence but not to each other.

The specific methodology employed to prepare the assay involved treating 1×10⁷ capture beads and 2×10⁷ reporter beads in 100 microgram per milliliter salmon sperm DNA for 1 hour at room temperature. This pretreatment will reduce non-covalent binding between the capture and reporter beads in the absence of target DNA. The capture beads were concentrated magnetically with the supernatant being removed. 100 microliters of the hybridization buffer (0.2M NaCl, 1 mM EDTA, 10 mM MgCl₂, 50 mM Tris-HCl, pH 7.5 and 5× Denhart's mix, 10 microgram per milliliter denatured salmon sperm DNA) were added and the beads were resuspended. Various concentration of target DNA ranging from 1, 10, 100 and 1000 femtomoles were added to the capture bead suspensions. The suspension was incubated while mixing at 37 degrees Celsius for 2 hours. The beads were magnetically concentrated and the supernatant containing unbound target DNA was removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated two times.

2×10⁷ reporter beads in 100 microliter hybridization buffer (0.2M NaCl, 1 mM EDTA, 10 mM MgCl₂, 50 mM Tris-HCl, pH 7.5 and 5× Denhart's mix, 10 microgram per milliliter denatured salmon sperm DNA) were then added to washed capture beads. The beads were resuspended and incubated while mixing at 37 degrees Centigrade for an additional 2 hours. After incubation, the capture beads were concentrated magnetically, and the supernatant containing unbound reporter beads were removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated two times.

After the final wash, the beads were resuspended in 20 microliters of binding buffer (50 mM Tris, 200 mM NaCl, 10 mM MgCl₂, 0.05% T-20, 1% BSA). 10 microliters was loading onto the disc that was prepared as described in 2.

2. Preparation of the Disc.

A gold disc was coated with maleic anhydride polystyrene. An amine DNA sequence complementary to the reporter probes (or capture agent) was immobilized onto discrete reaction zones on the disc. Prior to sample injection, the channels were blocked with a blocking buffer (50 mM Tris, 200 mM NaCl, 10 mM MgCl₂, 0.05% T-20, 1% BSA, 1% sucrose) to prevent non-covalent binding of the dual bead complex to the disc surface.

Alternatively, if the reporter beads are coated with streptavidin, a capture zone could be created with the capture agent such as BSA-biotin, which could be immobilized onto the disc (pretreated with polystyrene) by passive absorption.

3. Capture of Dual Bead Complex Structures on the Disc

10 microliters of the dual bead mixture prepared as described in part 1 was loaded into the disc chamber and the injection ports were sealed. To facilitate hybridization between the reporter probes on the reporter beads and the capture agents, the disc was centrifuged at low speed (less than 800 rpm) up to 15 minutes. The disc was read in the CD-reader at the speed 4× (˜1600 rpm) for 5 minutes. Under these conditions, the unbound magnetic capture beads were centrifuged away from the capture zone. The magnetic capture beads that were in the dual bead complex remained bound to the reporter beads in the capture zone.

4. Quantification of the Dual Bead Complex Structures.

The amount of target DNA captured could be enumerated by quantifying the number of capture magnetic beads and the number of reporter beads since each type of beads has a distinct signature.

### Example 2

1. Dual Bead Assay Multiplexing

In this example, the dual bead assay is carried out to detect 2 DNA targets simultaneously. The assay is comprised of 3 micron magnetic capture beads (Spherotech, Libertyville, Ill.). One population of the magnetic capture bead is coated with capture probes 1 which are complementary to the DNA target 1, another population of the magnetic capture beads is coated with capture probes 2 which are complementary to the DNA target 2. Alternatively, 2 different types of magnetic capture beads may be used. There are two distinct types of reporter beads in the assay. The two types may differ by chemical composition (for example silica and polystyrene) and/or by size. One type of reporter beads is coated with reporter probes 1, which are complementary to the DNA target 1. The other reporter beads are coated with reporter probes 2, which are complementary to the DNA target 2. Again, the capture probes and reporter probes are complementary to the respective targets but not to each other.

The specific methodology employed to prepare the dual bead assay multiplexing involved treating 1×10⁷ capture beads and 2×10⁷ reporter beads in 100 microgram per milliliter salmon sperm DNA for 1 hour at room temperature. This pretreatment will reduce the non-covalent binding between the capture and reporter beads in the absence of targets DNA. The capture beads were concentrated magnetically with the supernatant being removed. 100 microliters of the hybridization buffer (0.2M NaCl, 1 mM EDTA, 10 mM MgCl₂, 50 mM Tris-HCl, pH 7.5 and 5× Denhart's mix, 10 microgram per milliliter denatured salmon sperm DNA) were added and the beads were resuspended. Various concentration of target DNA ranging from 1, 10, 100 and 1000 femtomoles were added to the capture bead suspensions. The suspension was incubated while mixing at 37 degrees Centigrade for 2 hours. The beads were magnetically concentrated and the supernatant containing unbound target DNA was removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated two times.

2×10⁷ of each type of reporter beads in 100 ul hybridization buffer (0.2M NaCl, 1 mM EDTA, 10 mM MgCl₂, 50 mM Tris-HCl, pH 7.5 and 5× Denhart's mix, 10 microgram per milliliter denatured salmon sperm DNA) were then added to washed capture beads. The beads were resuspended and incubated while mixing at 37 degrees Centigrade for an additional 2 hours. After incubation, the capture beads were concentrated magnetically, and the supernatant containing unbound reporter beads were removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated two times.

After the final wash, the beads were resuspended in 20 microliters of binding buffer (50 mM Tris, 200 mM NaCl, 10 mM MgCl₂, 0.05% T-20, 1% BSA). 10 microliters was loading onto the disc that was prepared as described in 2.

2. Preparation of the Disc.

A gold disc was coated with maleic anhydride polystyrene as described previously. Distinct reaction zones were created for the 2 types of reporter beads. Each reaction zone consisted of amine DNA sequences complementary to the respective reporter probes (or capture agent). Prior to sample injection, the channels were blocked with a blocking buffer (50 mM Tris, 200 mM NaCl, 10 mM MgCl₂, 0.05% T-20, 1% BSA, 1% sucrose) to prevent non-covalent binding of the dual bead complex to the disc surface.

3. Capture of Dual Bead Complex Structures on the Disc.

10 microliters of the dual bead mixture prepared as described in part 1 was loaded into the disc chamber. The injection ports were sealed. To facilitate hybridization between the reporter probes on the reporter beads and the capture agents, the disc was centrifuged at low speed (less than 800 rpm) up to 15 minutes. The disc was read in the CD-reader at the speed 4× (˜1600 rpm) for 5 minutes. Under these conditions, the unbound magnetic capture beads were centrifuged to the bottom of the channels. The reporter beads bound to the capture zone via hybridization between the reporter probes and their complementary capture agent.

4. Quantification of the Dual Bead Complex Structures.

The amount of DNA targets 1 and 2 captured could be enumerated by quantifying the number of the respective reporter beads in the respective reaction zones.

### Example 3

The invention provides for the testing of various solid phases for efficient covalent conjugation of a probe. Referring to FIG. 29, various magnetic beads were evaluated for use in a dual bead assay. The dual bead assay is comprised of magnetic capture beads coated with a covalently attached capture probe; and fluorescent reporter beads coated with a covalently attached reporter probe. The capture probe and reporter probe were each 40 nucleotides in length and were complementary to the analyte of interest but not to each other. The capture probe and reporter probe each contained an amine group (NH₂) at the 5′ end and 3′ end of the molecule. The first step in the dual bead assay consists in selection of the beads used for covalent conjugation of probes. For the purpose of this experiment, the probe used for capture also contained a biotin group at the 3′ end of the molecule. Two types of magnetic beads were evaluated in this experiment.

1. Conjugation

Magnetic beads (1-2 μm) from Polysciences (Warrington, Pa.), magnetic beads (3 μm) from Spherotech (Libertyville, Ill.), fluorescent beads (1.8 μm) from Polysciences (Warrington, Pa.), and fluorescent beads (2.1 μm) from Molecular Probe (Eugene, Oreg.) were evaluated in this test. Approximately, 5×10⁸ beads were used per conjugation reaction. The beads were activated for 15 minutes in 1 ml of 0.05M MES buffer (2-N-morpholino-ethanesulfonic acid), pH 6.0, by the addition of 0.1 M EDC (1-Ethyl 3-3 dimethylaminopropyl carbodiimide-HCl). To quantify the amount of DNA probe binding non-covalently to the beads, a similar conjugation was carried out in the absence of the cross-linking agent EDC. After activation, 0.5 mmoles of amine capture probes was added. The conjugation was carried out for 2 to 3 hours at room temperature on a rotating mixer. The beads were then magnetically concentrated, and the supernatant was again removed. To estimate the amount of probes bound to the beads, the optical density at 260 nm of the supernatant could be measured before and after the conjugation. However, most of the times, only a small change in optical density was observed, which made the determination of the amount of probe bound to beads difficult and inaccurate.

After the conjugation, all unreactive carboxyl groups on the beads were blocked with the addition of 50 μL ethanolamine. The tubes were mixed for 30 minutes at room temperature. The supernatant was discarded. The beads were then mixed for 30 minutes in 1 ml of 10 mg/ml BSA in PBS to block any unspecific protein-binding site. The beads were then washed three times with PBS and resuspended in 500 μl of storage buffer (PBS with 10 mg/ml BSA, 5% glycerol, 0.1% NaN₃).

2. Determination of Covalent Conjugation Efficiency:

Typically, 1×10⁷ and 2×10⁷ magnetic beads were used in the determination of probe concentration. The beads were resuspended in 100 μl of binding buffer (2% BSA, 50 mM Tris-HCl, 145 mM NaCl, 1 mM MgCl₂, 0.1 mM ZnCl₂). The beads were then magnetically concentrated, and the supernatant was removed. The beads were resuspended in 100 μl of a solution of 700 ng/ml streptavidin-alkaline phosphatase (S-AP) (Pierce, Rockford, Ill.) and incubated for 1 hour at 37° C. During this step, streptavidin binds to the biotinylated probe. Following incubation with S-AP, the beads were magnetically concentrated, and the supernatant containing unbound S-AP was removed. The beads were washed three times in wash buffer (145 mM NaCl, 50 mM Tris, pH 7.5, 0.1% SDS, 0.05% Tween). Next, 100 μl of p-nitrophenyl phosphate (pNPP), a substrate for alkaline phosphatase, at a concentration of 3.7 mg/ml in 0.1M Tris-HCl, pH 10, and 2 mM MgCl₂ was added to the beads. The product formed from the pNPP substrate is yellow and has a strong absorbance at 405 nm. The optical density at 405 nm was proportional to the amount of probes bound to the beads.

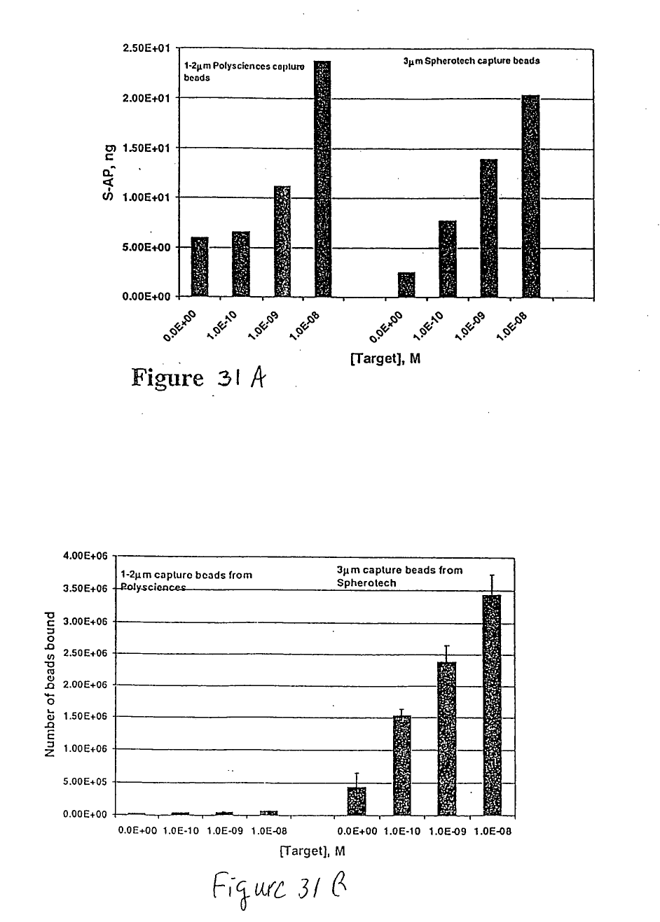

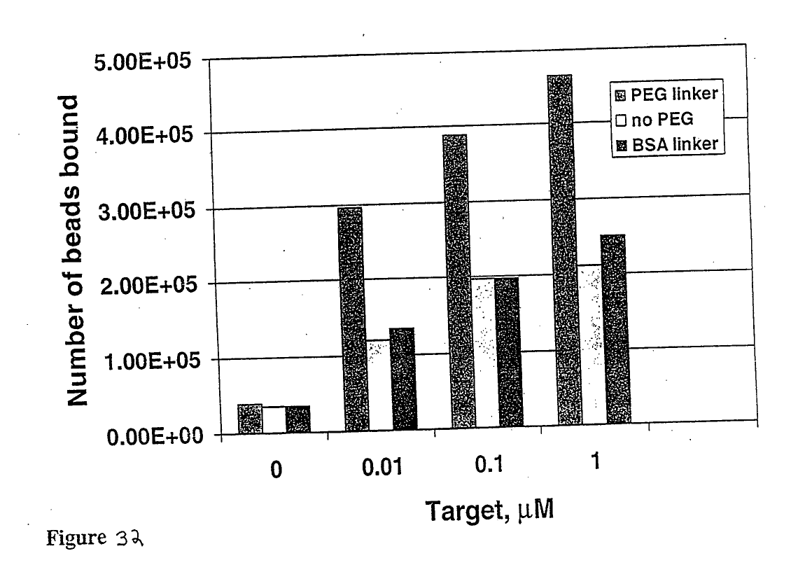

Results of the experiment are presented in FIGS. 29A, 29B, 31A and 31B. Referring to FIG. 29A, up to 90% of the probe that bound to the 1-2 μm magnetic beads from Polysciences were non-covalently bound, as compared to 15-25% of non-covalently bound probe on the 3 μm magnetic beads from Spherotech. Results of experiments conducted on different fluorescent-type beads were substantially similar (FIG. 29B). These results indicate that to obtain covalent linkages for improved specificity, solid phases should be screened. For example, for a dual bead assay, the 3 μm magnetic beads would be much more suitable as capture beads than the 1-2 μm magnetic beads. Referring to FIGS. 31A and 31B, data showing a correlation between the covalent conjugation efficiency and the sensitivity of the dual bead assay is presented. These results indicate that with a higher covalent conjugation efficiency, the dual bead assay is more sensitive and specific. Covalent linkage of the probe to a solid phase provides an interaction of sufficient strength for the dual bead assay, whereas a non-covalent linkage does not.

3. Heat Treatment Removal of Non-Covalently Bound Probe

If 100% covalent conjugation efficiency is desired for the dual bead assay, following conjugation, the non-covalently bound probes could be selectively removed by heat treatment of the beads. For this purpose, up to 5×10⁷ beads were resuspended in 100 μl of binding buffer (2% BSA, 50 mM Tris-HCl, 145 mM NaCl, 1 mM MgCl₂, 0.1 mM ZnCl₂), and the solution was heated at 70° C. for 10 minutes. The beads were then magnetically concentrated, and the supernatant was removed. The beads were washed three times in wash buffer (145 mM NaCl, 50 mM Tris, pH 7.5, 0.1% SDS, 0.05% Tween) and resuspended in 100 μl of binding buffer (2% BSA, 50 mM Tris-HCl, 145 mM NaCl, 1 mM MgCl₂, 0.1 mM ZnCl₂) or any buffer suitable for the assay of interest.

### Example 4

Experiments were also done to evaluate the use of double-stranded DNA as a probe to minimize non-covalent binding to a solid phase.

1. Formation of the Double-Stranded DNA:

The capture probe utilized was 40 nucleotides in length and contained an amine group (NH₂) at the 5′ end and several linker groups (polyethylene glycol) (FIG. 12). The complementary probe was also 40 nucleotides in length and contained a biotin group at the 5′ end. A hybridization reaction was carried out with an excess of complementary probe under stringent conditions at 37° C. After the hybridization, the reaction mixture was run on a non-denaturing gel to insure the formation of the hybrid. Under the conditions used, the hybridization reaction was complete.

2. Conjugation of the Double-Stranded DNA Probe Onto Beads

The conjugation of the double-stranded DNA was carried out as previously described for single stranded DNA in Example 3. Briefly, 5×10⁸ beads were used per conjugation. The beads were activated for 15 minutes in 1 ml of 0.05 M MES (2-N-morpholino-ethanesulfonic acid) buffer, pH 6.0, by the addition of 0.1M EDC (1-Ethyl 3-3 dimethylaminopropyl carbodiimide-HCl). To quantify the amount of DNA probe binding non-covalently to the beads, a similar conjugation was carried out in the absence of the cross-linking agent EDC. After activation, 0.5 nmoles of amine capture probes was added, and the conjugation was carried out for 2 to 3 hours at room temperature on a rotating mixer.

The beads were then magnetically concentrated, and the supernatant was removed. An attempt was made to estimate the amount of probe bound to the beads by measuring optical density at 260 nm of the supernatant as previously described.

After the conjugation step, all unreactive carboxyl groups on the beads were blocked by the addition of 50 μL ethanolamine, and the sample was mixed for 30 minutes at room temperature. The beads were separated from the supernatant and then mixed for 30 minutes in 1 ml of 10 mg/ml BSA in phosphate buffered saline (PBS) to block any unspecific protein-binding sites. The beads were then washed three times with PBS and resuspended in 500 μl of storage buffer (PBS with 10 mg/ml BSA, 5% glycerol, 0.1% NaN₃).

3. Determination of Covalent Conjugation Efficiency

The biotin group on the 5′ end of the complementarty probe allows for the easy quantification of double-stranded probe bound to the beads covalently and non covalently. Conjugation efficiency was determined as described in Example 3. Results of these experiments are presented in FIG. 30B, which clearly indicates that there is a much higher ratio of covalently bound probe to non-covalently bound probe when double-stranded DNA is utilized.

4. Use of Heat Treatment to Separate the Complementary Strand from the Capture Probes:

To capture the target of interest in the dual bead assay, the complementary probe can be easily separated from the capture probe by heat treatment. For this purpose, up to 5×10⁷ beads were resuspended in 100 μl of binding buffer (2% BSA, 50 mM Tris-HCl, 145 mM NaCl, 1 mM MgCl₂, 0.1 mM ZnCl₂). The solution was heated at 70° C. for 10 minutes. The beads were then magnetically concentrated and the supernatant was removed. The beads were washed three times in wash buffer (145 mM NaCl, 50 mM Tris, pH 7.5, 0.1% SDS, 0.05% Tween) and resuspended in 100 μl of binding buffer (2% BSA, 50 mM Tris-HCl, 145 mM NaCl, 1 mM MgCl₂, 0.1 mM ZnCl₂) or any buffer suitable for the assay of interest capture beads in the dual bead assay.

### Example 5

Experiments were also conducted to test the use of linkers to increase the accessibility and rigidity of the probes attached to a solid phase. In these experiments, the capture and reporter probes were 40 nucleotides in length. These synthetic nucleotide sequences were specific to the analyte of interest. Various linkers can be added at the end of a probe to make it longer and more rigid. In this example, 3 polyethylene glycol moieties were added to the 5′ end of the capture probe and 3′ end of the reporter probe via EDC coupling. The structure of the capture probe was: 5′-NH₂-PEG-PEG-PEG-CCA-GTG-AAT-TCG-AGC-TCG-GTA-CCC-GGG-GAT-CCT-CTA-GAG-T-3′ (SEQ ID NO:1). The structure of the reporter probe was: 5′-CTT AGT CTT TAG ATG CAA GCT TGG CGT AAT CAT GGT CAT A PEG PEG PEG-NH₂ 3′ (SEQ ID NO:2).

After coupling, the tagged probes were purified by HPLC. Alternatively, the probes could also be coupled to BSA via EDC coupling. Results showed that when PEG linkers introduced into the capture probes improved the sensitivity of the dual bead assay significantly (FIG. 32).

1. A method of evaluating the suitability of a solid phase as a binding surface, the method comprising:
(i) selecting a test solid phase;
(ii) binding a probe or capture agent to a first and a second sample of the test solid phase, in a first and a second binding reaction, respectively, wherein said first binding reaction comprises a cross-linking agent and said second binding reaction lacks a cross-linking agent;
(iii) determining the amount of probe or capture agent bound to the first sample of the test solid phase;
(iv) determining the amount of probe or capture agent bound to the second sample of the test solid phase; and
(v) comparing the amount of probe or capture agent bound to the first sample of the test solid phase to the amount of probe or capture agent bound to the second sample of the test solid phase, wherein if a ratio of the amount of probe or capture agent bound to the second sample of the test solid phase to the amount of probe or capture agent bound to the first sample of the test solid phase is less than a threshold value, the test solid phase is suitable as a binding surface.

2. The method of claim 1, wherein the threshold value is approximately 0.2.

3. The method of claim 1, wherein the solid phase comprises a bead.

4. The method of claim 3, wherein the bead comprises a magnetic bead.

5. The method of claim 1, wherein the solid phase comprises a biodisc surface.

6. The method of claim 1, wherein the probe or capture agent comprises a nucleic acid.

7. The method of claim 6, wherein the nucleic acid is double stranded.

8. The method of claim 6, wherein the probe further comprises a linker.

9. The method of claim 8, wherein the linker comprises at least one polyethylene glycol moiety.

10. The method of claim 1, wherein the probe or capture agent comprises a protein.

11. The method of claim 10, wherein the probe further comprises a linker.

12. The method of claim 11, wherein the linker comprises at least one polyethylene glycol moiety.

13. The method of claim 1, wherein the solid phase is attached to a biodisc.

14. The method of claim 1, wherein the solid phase is within but not attached to a biodisc.

15. The method of claim 1, wherein the cross-linking agent comprises 1-ethyl-3-(3-dimethylaminopropyl) carbodiimide.

16. A method of evaluating the suitability of a solid phase as a binding surface, the method comprising:
selecting a test solid phase;
conjugating the test solid phase with a probe or capture agent;
washing the test solid phase with a wash solution;
heat treating the the test solid phase, thereby removing non-covalently bound probe or capture agent, and;
determining the percentage of probe or capture agent bound covalently to the test solid phase, wherein if the amount of probe or capture agent bound covalently to the test solid phase is greater than a threshold value, the test solid phase is suitable as a binding surface.

17. The method of claim 16, wherein the threshold value is approximately 50%.

18. The method of claim 16, wherein the threshold value is approximately 80%.

19. The method of claim 16, wherein the solid phase comprises a bead.

20. The method of claim 19, wherein the bead comprises a magnetic bead.

21. The method of claim 16, wherein the solid phase comprises a biodisc surface.

22. The method of claim 16, wherein said conjugation is performed in the presence of a cross-linking agent.

23. The method of claim 22, wherein said cross-linking agent comprises 1-ethyl-3-(3-dimethylaminopropyl) carbodiimide.

24. The method of claim 16, wherein said wash solution is a conjugate dilution buffer.
